(19) United States
(12) Patent Application Publication
(10) Pub. No.: US 2021/0340155 A1
(43) Pub. Date: Nov. 4, 2021

(54) Substituted (4-Hydroxyphenyl)Cycloalkane and (4-Hydroxyphenyl)Cycloalkene Compounds and Uses Thereof as Selective Agonists of the Estrogen Receptor Beta Isoform for Enhanced Memory Consolidation

(71) Applicants: Marquette University, Milwaukee, WI (US); Concordia University, Inc., Mequon, WI (US); UWM Research Foundation, Inc., Milwaukee, WI (US)

(72) Inventors: William A. Donaldson, Milwaukee, WI (US); Daniel S. Sem, New Berlin, WI (US); Karyn M. Frick, Milwaukee, WI (US)

(21) Appl. No.: 16/498,122

(22) PCT Filed: Mar. 30, 2018
(86) PCT No.: PCT/US2018/025342

§ 371 Date: Sep. 26, 2019

Related U.S. Application Data
(60) Provisional application No. 62/478,758, filed on Mar. 30, 2017.
(60) Provisional application No. 62/572,932, filed on Oct. 16, 2017.

(51) Int. Cl. C07D 493/08; A61P 25/28; C07C 35/21

(52) U.S. Cl. CPC C07D 493/08; C07C 35/21; A61P 25/28

(57) ABSTRACT

Disclosed are substituted (4′-hydroxylphenyl)cycloalkane compounds and substituted (4′-hydroxylphenyl)cycloalkene compounds and there use as selective agonists of the estrogen receptor beta isoform (ERβ). The disclosed compounds may be formulated as pharmaceutical compositions and administered for treating diseases associated with ER activity, such as neurological, psychiatric, and/or cell proliferative diseases and disorders as well as for enhancing memory consolidation in subjects in need thereof.

### CROSS-REFERENCE TO RELATED PATENT APPLICATIONS

The present application claims the benefit of priority under 35 U.S.C. § 119(e) to U.S. Provisional Application No. 62/572,932, filed on Oct. 16, 2017 and U.S. Provisional Application No. 62/478,758, filed on Mar. 30, 2017, the contents of which are incorporated herein by reference in their entireties.

### STATEMENT REGARDING FEDERALLY SPONSORED RESEARCH OR DEVELOPMENT

This invention was made with government support under R15GM118304 awarded by the National Institute of General Medical Sciences and R01DA038042 awarded by the National Institute on Drug Abuse. The Government has certain rights in this invention.

### FIELD OF THE INVENTION

The field of the invention relates to compounds that function as ligands for estrogen receptors (ERs). In particular, the field of the invention relates to substituted (4′-hydroxyphenyl)cycloalkane compounds and (4′-hydroxyphenyl)cycloalkene compounds that are specific agonists for the estrogen receptor beta (ERβ) and the use of such compounds in pharmaceutical compositions for treating diseases and disorders associated with ER activity in enhancing memory consolidation.

### BACKGROUND

Estrogens are important regulators of many physiological processes that include reproduction, cognition, cardiovascular health, and bone metabolism.⁶⁶ Based on their widespread role in a number of physiological processes, estrogens have been implicated in a number of diseases and disorders which include cell proliferative diseases and disorders (e.g., breast cancer, ovarian cancer, endometrial cancer, colorectal cancer, and prostate cancer), neurodegenerative diseases and disorders, cardiovascular disease, and osteoporosis to name a few.⁶⁶ In many of these diseases and disorders, estrogen mediates its effects through the estrogen receptors (ERs).

The ERs exist in 2 main forms, ERα and ERβ, which have different tissue expression patterns.⁶⁷ ERα and ERβ are encoded by separate genes, ESR1 and ESR2, respectively, found at different chromosomal locations, and numerous mRNA splice variants exist for both ERα and ERβ.⁶⁸ Because of their role in estrogen-related diseases, ERα and ERβ have been targeted for development of specific ligands that modulate their activities. The ligand specificity of ERα and ERβ differ, and a ligand that binds and functions as an agonist or antagonist for ERα may or may not bind and function as an agonist or antagonist for ERβ.

ERα and ERβ agonists have a wide range of biological effects that implicate disease such as cancer and disorders of the central nervous system (CNS). 17β-estradiol (E₂) is a critical modulator of hippocampal synaptic plasticity and hippocampal-dependent memory formation in male and female rodents.⁶ E₂ levels decrease in both sexes as people age, but drop much more precipitously in women during the menopausal transition. ERβ is the predominant ER isoform in the hippocampus and plays an important role in mediating estradiol's effects on neural plasticity and neuroprotection, which could be pivotal during aging and in Alzheimer's disease (AD). For example, overexpression of ERβ in a rat model of AD significantly reduced hippocampal AD pathology and improved learning and memory.⁷ Moreover, specific alleles of the gene for ERβ (Esr2), but not ERα, are associated with decreased AD risk in men and women,⁸ supporting ERβ as a putative drug target for AD.

ERβ agonists in particular have a number of promising clinical applications¹. Current ERβ agonist drug lead molecules possess a phenolic ring, with varying substituted aromatic ring systems on the other half of the molecule, typically comprising another phenolic or indole-like ring systems (FIG. 1a). One of these, WAY-200070 (benzoxazole), has shown efficacy as an anxiolytic/antidepressant and has 68-fold selectivity for ERβ over ERα.¹⁻³ Some ERβ agonists have progressed into human clinical trials for different disease indications, ranging from schizophrenia (Eli Lilly; NCT01874756), to Fragile-X syndrome (Parc de Salut Mar; NCT01855971), to memory loss and hot flashes (National Institutes on Aging; NCT01723917).⁴ Studies presented herein focus on one of the more promising new clinical applications of ERβ agonists, for treating neuronal symptoms caused by estrogen deficiency in menopause, as illustrated in animal model studies using diaryl propionitrile (DPN).⁵

APOE4 is the most well established genetic risk factor for Alzheimer's disease (AD). Women with the APOE4 genotype are 2-4 times more likely to develop AD than women without APOE4 or than men of any other APOE genotype.⁹⁻¹¹ APOE4 carriers are also much more likely to show symptoms of anxiety and depression.¹² A major contributor to these risks in women is menopausal estrogen loss, as estrogens are neuroprotective for brain regions like the hippocampus and cortex that mediate cognitive function and deteriorate in AD.¹³ As such, drugs that facilitate estrogen-mediated effects on cognition, like the selective ERβ agonists (SERBAs) being developed herein, may reverse memory loss and alleviate anxiety and depression in aging females. But, estrogen-based hormone replacement therapy is associated with increased risk of various diseases (thought to be associated with ERα agonist activity), including breast cancer (esp. lobular) as well as stroke, gallbladder disease and venous thromboembolism¹⁴⁻¹⁸. Accordingly, any ERβ agonist therapeutic should be selective for ERβ over ERα agonist activity.

Thus, new ligands for estrogen receptors are desirable. In particular, new ligands that exhibit selective agonist or antagonist activity for ERβ versus ERα are desirable. These new ligands should be suitable for treating diseases and disorders associated with ER activity, such as cell proliferative diseases and disorders or psychiatric diseases and disorders. Recently, we reported a novel ERβ agonist that was more selective for ERβ versus ERα activation than previously reported clinical candidates¹⁹. This ERβ agonist was in a unique structural class, comprised of a phenol ring tethered to a 4-hydroxymethyl-cycloheptane ring system. However, the presence of the 4-substituted cycloheptane ring presents synthetic and stereochemistry challenges, making it less desirable as a drug lead.

Herein is reported the optimization and characterization of a related class of molecules, comprised of a 4-hydroxymethyl-cyclohexane ring tethered to a phenol ring, making it an A-C estrogen that closely resembles the naturally occurring estrogen molecule, but lacks the B and D rings (FIGS. 1b-d). Whereas A-CD estrogens have been widely studied and reported to have up to 15-fold selectivity for ERβ,¹⁶⁻²³ the simpler A-C estrogens reported herein show even higher selectivity for ERβ over ERα. These A-C estrogens represent a surprisingly simple yet novel class of isoform selective ERβ agonists, with potential for treating age-related memory decline in post-menopausal women.

### SUMMARY

Disclosed are substituted (4′-hydroxylphenyl)cycloalkane compounds and (4′-hydroxylphenyl)cycloalkene compounds and their use as selective agonists of the estrogen receptor beta (ERβ). The disclosed compounds may be formulated as pharmaceutical compositions and administered to treat diseases associated with ER activity.

In some embodiments, the disclosed compounds have a Formula I or a hydroxy-protected form thereof:

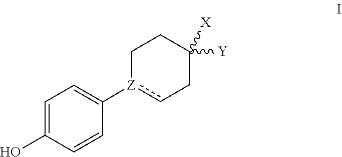

wherein:
(a) Z is a carbon atom;
(b) X is selected from the group consisting of hydrogen, hydroxyl, alkyl, hydroxyalkyl, amino, and aminoalkyl; and
(c) Y is selected from the group consisting of hydrogen, hydroxyl, alkyl, and hydroxyalkyl; or Y is —CH₂CH₂— or —OCH₂— and Y and Z form a bridge; or X and Y together form alkylidenyl, carboxyalkylidenyl, esteralkylidenyl, hydroxyalkylidenyl, hydroxyalkylalkylidenyl, aminoalkylidenyl, oxo, or oxime.

The disclosed compounds may include 4-substituted-(4′-hydroxyphenyl)cyclohexane compounds. For example, the disclosed compounds may have a Formula Ia:

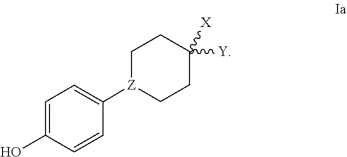

where X, Y, and Z are as defined for Formula I.

The disclosed compounds include the compound 4-hydroxymethyl-(4′hydroxyphenyl)-cyclohexane and in particular the enantiomer:

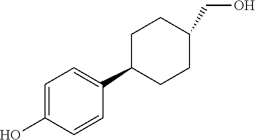 [SMILES: OC[C@H]1CC[C@H](c2ccc(O)cc2)CC1]

otherwise referred to herein as “ISP358-2”.

The disclosed compounds may include 4-substituted-(4′-hydroxyphenyl)cyclohexene compounds. For example, the disclosed compounds may have a Formula Ia(i):

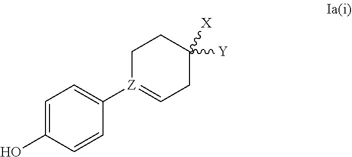

where X, Y, and Z are as defined for Formula I.

The disclosed compounds may be used to prepare and formulate pharmaceutical compositions. As such, also disclosed herein are pharmaceutical compositions comprising an effective amount of any of the compounds disclosed herein, or pharmaceutically acceptable salts of any of the compounds disclosed herein, together with a pharmaceutically acceptable excipient, carrier, or diluent.

The disclosed compounds may be used for preparing a medicament for treating a disease or disorder associated with estrogen receptor 13 (ERβ) activity, and in particular, a disease or disorder that may be treated with an agonist of ERβ. As such, the disclosed compounds may exhibit ERβ agonist activity, and preferable the compounds exhibit specificity as ERβ agonists versus activity as ERβ antagonists and/or versus activity as estrogen receptor α (ERα) agonists or activity as ERα antagonists. The disclosed compounds may be formulated for use in treating psychiatric or neurological diseases or disorders. In particular, the disclosed compounds may be formulated for use in treating a subject in need of enhanced memory consolidation, for example, enhanced memory consolidation under low estrogen conditions observed in post-menopausal women.

### BRIEF DESCRIPTION OF THE DRAWINGS

FIG. 1. Estrogen Receptor Agonist Structures. (a) Previously reported ERβ agonists. Figure adapted from [35]. (b) Structure of ISP358-2, (c) estradiol (E₂), and (d) ISP538-2 overlaid on top of E₂, to illustrate the similarity to the naturally occurring estrogen.

FIG. 2. Estrogen Receptor Binding Assays. (a) TR-FRET binding assay for binding to the ligand binding domain (LBD) of ERβ. (b) ISP358-2 binding to the LBDs of ERβ and ERα. ISP-358-2 has modest 12-fold selectivity for ERβ (IC₅₀=24+5 nM) relative to ERα (IC₅₀=289±92 nM) in this assay.

FIG. 3. Nuclear Hormone Receptor Specificity Assay for ISP358-2. (a) Agonist activity was measured in the GeneBLAzer™ cell-based transcriptional activation assay at three concentrations of ISP358-2, using chimeric nuclear hormone receptors (NRs) comprised of the relevant NR ligand binding domains (LBDs), and the DNA binding domain (DBD) from GAL4. Assay was with 9 different NRs: Androgen Receptor (AR), Glucocorticoid Receptor (GR), Mineralocorticoid Receptor (MR), Peroxisome Proliferator-Activated Receptor (PPARδ), Progesterone Receptor (PR), Thyroid Hormone Receptor (TRβ), and Vitamin D Receptor (VDR). ISP358-2 has high selectivity for binding to ER relative to other nuclear receptors (NRs). (b) Agonist activity dose-response curve (open symbols) in the GeneBLAzer™ assay for ERβ and ERα, showing a modest 2.6-fold selectivity for ERβ (IC₅₀=357±26 nM) over ERα (IC₅₀=930±69 nM). Data from panel (a) are included for comparison (closed symbols).

FIG. 4. Specificity Assay for ISP-358-2 Binding in a Coactivator Assay. (a) This assay measures recruitment of a labeled coactivator peptide to the ERα or ERβ LBD, induced by the binding of an ER agonist (ISP358-2, in this case). The coactivator peptide is derived from the PPARγ coactivator protein 1a. Figure is adapted from the ThermoFisher manual. (b) Chemical structure of ISP358-2. (c) ISP358-2 dose-response curve in the coactivator assay, giving an IC₅₀ of 161+15 nM for ERβ and 2,940+390 nM for ERα, giving an ERβ selectivity of 15-fold.

FIG. 5. Specificity Assay for ISP358-2 in Cell-based Assays. (a) ERβ and (b) ERα agonist activity, based on activation of transcription by a full length estrogen receptor. (c) ERβ and (d) ERα antagonist activity, based on inhibition of estradiol-induced transcription by an antagonist compound. Average ERβ agonist potency is 27±4 nM (data here has IC₅₀ of 31±7) in panel (a), and ERα agonist potency is 20,400±860 nM. This gives an ERβ selectivity of ≅750-fold. No measurable antagonist activity was observed for ERβ or ERα at concentrations of ISP358-2 up to 10 μM, in panels (c) and (d).

FIG. 6. Cytochrome P450 inhibition by ISP358-2. Inhibition of CYP450 activity (Promega P450-Glo™ assay) by ISP358-2 for: CYP2D6, CYP3A4 (IC₅₀=89±18 μM), (c) CYP1A2 and (d) CYP2C9 (IC₅₀=34±4.7 μM).

FIG. 7. Structural Analysis of ISP358-2. (a) Crystal structure (Ortep rendering) of ISP358-2, showing the trans stereochemistry of the cyclohexane ring. (b) Docked structure of ISP358-2 in the ERα binding pocket, showing interactions with active site residues, including hydrogen bonding with Arg394, Glu353 and His524. (c) Same as panel b, but for ISP358-2 bound to ERβ. Hydrophobic interactions in ERβ are observed between the phenol ring of ISP358-2 and Phe356, Phe355 and Met 340; and, between the cyclohexane ring and Leu476, Ala302, Ile373 and Leu298. Docking energy is −7.6 kcal/mol in ERα and −8.0 kcal/mol in ERβ.

FIG. 8. Behavioral Assays. (a) Overview of the OR and OP testing procedures. When infused into the DH, DPN at the 100 pg and 1 ng/hemisphere doses of ISP358-2, significantly increased time was spent with the moved (b) or novel (c) object relative to chance (15 s; *p<0.05; **p<0.01) and vehicle (#p<0.05; ##p<0.01), suggesting that ISP358-2 enhanced memory consolidation to a similar extent as the positive control DPN. When injected IP, DPN and 0.5 mg/kg ISP358-2 enhanced memory consolidation in the OP (d) and OR (e) tests (###p<0.001). Five mg/kg ISP358-2 also enhanced OP memory consolidation. Similarly, oral gavage treatments of 0.5 and 5 mg/kg ISP358-2 enhanced memory consolidation in the OP (f) and OR (g) tests. Panel (a) adapted from [38].

FIG. 9. ERβ Binding Assay. TR-FRET binding assay for binding to the ligand binding domain (LBD) of ERβ, including E2 and ERβ agonist DPN as controls.

FIG. 10. Cell-based assays comparing ISP358-2 to known compounds. Estrogen receptor agonist activity, based on transcription by the full-length estrogen receptor. (a) With ERα IC₅₀ values are 0.31±0.03 nM for E2, 2,300±86 nM for DPN, 2,103±414 nM for WAY 200070 and 18,615±939 for ISP358-2. (b) With ERβ IC₅₀ values are 0.022±0.005 nM for E2, 1.1±0.12 nM for DPN, 1.5±0.57 nM for WAY 200070, and 23±8 nM for ISP358-2.

FIG. 11. Assessment of in vitro druggability parameters. (a) Nephelometry indicates good solubility of 15:16. Note: Any compound with a nephelometry inflection point greater than 50 μM is considered soluble.¹ (b) hERG assay of ISP358-2 shows only 13% inhibition at 100 μM, suggesting no significant hERG activity.

FIG. 12. In vivo correlation of behavioral effect and ERβ levels. As shown in FIG. 8, administration of DPN or ISP358-2 via oral gavage enhanced spatial memory consolidation in mice ovariectomized within one month of OP testing. However, if treatment is delayed until 4 months after ovariectomy (ovx), then neither DPN nor ISP358-2 affected memory (panel a). Western blot analyses of ERα and ERβ levels in dorsal hippocampal tissue from these mice indicate that ERβ (but not ERα) levels are reduced 5 months after ovx relative to 2 months after ovx (panels b,c), suggesting that the lack of effect of DPN and ISP358-2 on memory in long-term ovx mice results from a reduction in ERβ levels. These data also suggest specific effects of the compounds on ERβ; because, if either compound acted via ERα, then they should have been able to enhance memory 4-5 months after ovx, given that ERα levels were not decreased at this time. The fact that neither affected memory 4 months after ovx is consistent with our hypothesis that ISP358-2, like DPN, is selective for ERβ over ERα in vivo.

FIG. 13. Tissue pathology analysis for ISP358-2. (a) Tissue samples analyzed were in 4 groups, each with 5 mice. The labels are ordered as V-D-L-H: Vehicle (V), DPN (D), 0.5 mg/kg ISP358-2 (L), and 5 mg/kg ISP358-2 (H). (b) Representative images of hematoxylin and eosin (H&E) stained tissue collected from vehicle control (A-C), DPN-treated (D-F), low dose ISP358-2 0.5 mg/kg treated (G-I), and high dose ISP358-2 5.0 mg/kg treated (J-L) animals. Shown are examples of portal vein and hepatic duct (A, D, G, J), glomeruli and tubules (B, E, H, K), and myocytes from the interventricular septum (C, F, I, L). Histological abnormalities were not detected in the control animal or in any of the treatment groups

FIG. 14. Bloodwork panel for tissue pathology analysis for ISP358-2. Bloodwork hematology analysis was performed by Animal Reference Pathology, LLC in Salt Lake City, Utah (animalreferencepathology.com). Reference ranges are from Charles River Laboratories for CrL:Wi (Han) female 8-16 week old rats. Blood samples were taken via cardiac puncture at the same time point and treatment conditions as in FIG. 13. Ovariectomized 9 week-old (n-8) mice were injected i.p. with vehicle, DPN, 0.5 mg/kg ISP358-2, or 5 mg/kg ISP358-2. In some cases hemolysis occurred, likely due to the blood collection procedure, which precluded analysis for those samples, thus, samples sizes in the analysis were 6-8. The mean and standard error of the mean of each group is listed in the table, along with outcomes from one-way ANOVA statistical analysis. In all cases of a significant ANOVA, Fisher's posthoc tests showed that the vehicle group was significantly different from all drug groups (p<0.05). The hematology samples were analyzed by a Sysmex XT-2000iV using veterinary software and the rat species setting.³ The reagents for individual assays were sourced from either Seimens, RandOx or Sekisui.

FIG. 15. MTT assay for proliferation of MCF-7 cells. Cells were seeded into 96-well plates and incubated for 24 hr before treatment was applied. All wells contained 0.1% DMSO, which does not impact proliferation significantly.² Compounds to be tested were dissolved in media, applied to cells, and cells were incubated an additional 24 hr, at which point the MTT (3-(4,5-dimethylthiazol-2-yl)-2,5-diphenyltetrazolium bromide) assay was done. Absorbance values were converted to cell counts using a standard growth curve. Treatments were: (a) E2, (b) ISP358-2, and (c) DPN. * indicate significantly different cell counts compared to the untreated control (Students T-test, p value <0.05).

FIG. 16. Purity analysis of ISP358-2 (16). ¹H NMR (400 MHz) spectra for: (a) a mixture of 16/15 (ca. 2:1 ratio) and (b) for the material which was sent for combustion analysis (16:15>98:2). The stereochemical configuration for 16 (aka ISP358-2) was confirmed by x-ray crystallography. Spectra show only the range from 4.0-3.0 ppm, for clarity in comparison of the signals for the CH₂OH (also contains the solvent peak used as reference @ 3.31 ppm).

### DETAILED DESCRIPTION

The present invention is described herein using several definitions, as set forth below and throughout the application.

Unless otherwise specified or indicated by context, the terms “a”, “an”, and “the” mean “one or more.” For example, “a substitution” should be interpreted to mean “one or more substitutions.” Similarly, “a substituent group” should be interpreted to mean “one or more substituent groups.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

As used herein, a “subject in need thereof” may include a human or a non-human animal. The term “subject” may be used interchangeably with the terms “individual” or “patient.”

As used herein, a “subject in need thereof” may include a subject in need of treatment with an agonist of the estrogen receptor beta isoform (ERβ). A subject in need of treatment with an agonist of ERβ may include a subject having subject having a disease or disorder associated with ERβ activity. Diseases and disorders associated with ERβ activity may include, but are not limited to, cell proliferative diseases and disorders (e.g., cancers such as breast cancer, ovarian cancer, and endometrial cancer), psychiatric diseases and disorders (e.g., depression, anxiety, and schizophrenia), neurodegenerative diseases or disorders (e.g., Alzheimer” s disease including APOE4 associated Alzheimer's disease), memory decline (e.g., memory decline observed under low estrogen conditions as those observed in post-menopausal women), bone metabolic diseases or disorders (e.g. osteoporosis), metabolic diseases or disorders (e.g., obesity or insulin resistance), and cardiovascular diseases or disorders.

A subject in need thereof may include a subject exhibiting low estrogen (i.e., estradiol) serum levels. A subject exhibiting low estrogen serum levels may be exhibiting low estrogen serum levels associated with menopause (e.g., as observed in post-menopausal women). A subject exhibiting low estrogen serum levels may include a subject exhibiting estrogen serum levels of less than about 60 pg/ml, 55 pg/ml, 50 pg/ml, 45 pg/ml, 40 pg/ml, 35 pg/ml, 30 pg/ml, 25 pg/ml, 20 pg/ml, 15 pg/ml, 10 pg/ml, 5 pg/ml, or less, or exhibiting estrogen serum levels within a range bounded by any of these values (e.g., within a range of 15-60 pg/ml).

A subject in need thereof may include a subject exhibiting low estrogen serum levels associated with the subject having been administered a therapy and/or treatment which reduces estrogen serum levels and/or estrogen activity in the subject. A subject in need thereof may include a subject undergoing therapy for cancer treatment or therapy after cancer treatment (e.g., therapy for breast cancer treatment and/or therapy after breast cancer treatment). A subject in need thereof may include a subject undergoing hormone therapy (e.g., hormone therapy for cancer such as breast cancer) and/or a subject undergoing hormone replacement therapy (e.g., hormone replacement therapy after treatment for cancer such as breast cancer). A subject in need thereof may include a subject undergoing treatment with drugs that may include, but are not limited to, tamoxifen, toremifene (Fareston), fulvestrant (Faslodex), aromatase inhibitors (e.g., letrozole (Femara), anastrozole (Arimidex), and exemestane (Aromasin)), lutenizing hormone-releasing hormone (LHRH) analogs (e.g., goserelin (Zoladex) and Leuprolide (Lupron)). A subject in need thereof may include a subject having undergone an oophorectomy and/or a hysterectomy.

Disclosed are substituted (4′-hydroxylphenyl)cycloalkane compounds and (4′-hydroxylphenyl)cycloalkene compounds and there use as selective agonists of the estrogen receptor beta isoform (ERβ). Several compounds of this class of compounds have been previously described in U.S. Patent Pub. No. 2016/0340279 to Donaldson et al., the contents of which are incorporated herein by reference in its entirety. The disclosed compounds may alternatively be referred to as substituted 4-cycloalkylphenol compounds or p-cycloalkyl substituted phenol compounds that include one or more substitutions on the cycloalkyl substituent, which cycloalkyl substituent preferably is a cyclohexyl substituent.

In some embodiments, the disclosed compounds include one or more substitutions on the 4-carbon of the cycloalkyl substituent and have a Formula I:

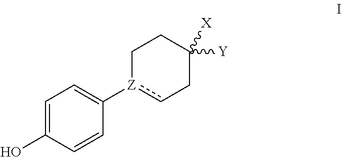

where:
(a) Z is a carbon atom;
(b) X is selected from the group consisting of hydrogen, hydroxyl, alkyl, hydroxyalkyl, amino, and aminoalkyl; and
(c) Y is selected from the group consisting of hydrogen, hydroxyl, alkyl, and hydroxyalkyl; or Y is —CH₂CH₂— or —OCH₂— and Y and Z form a bridge; or X and Y together form alkylidenyl, carboxyalkylidenyl, esteralkylidenyl, hydroxyalkylidenyl, hydroxyalkylalkylidenyl, aminoalkylidenyl, oxo, or oxime.

The alkyl moiety of the X or Y substituents may be a C(1-6)alkyl. In certain embodiments, the alkyl moiety may be a C(1-3)alkyl. The hydroxyalkyl moiety of the X or Y substituents may be a hydroxyl-C(1-6)alkyl. In certain embodiments, the hydroxyalkyl moiety may be a hydroxyl-C(1-3)alkyl. The aminoalkyl moiety of the X or Y substituents may be a amino-C(1-6)alkyl. In certain embodiments, the aminoalkyl moiety may be a amino-C(1-3)alkyl.

The alkyl portion of the carboxyalkylidenyl, esteralkylidenyl, hydroxyalkylidenyl, or aminoalkylidenyl moieties may be a C(1-6)alkyl. In certain embodiments, alkyl portion of the carboxyalkylidenyl, esteralkylidenyl, hydroxyalkylidenyl, or aminoalkylidenyl may be a C(1-3)alkyl. For example, the carboxyalkylidenyl may be a carboxy-C(1-6)alkylidenyl or carboxy-C(1-3)alkylidenyl; the esteralkylidenyl may be a C(1-6)alkyl-ester-C(1-6)alkylidenyl or C(1-3)alkyl-ester-C(1-3)alkylidenyl; the hydroxyalkylidenyl may be a hydroxy-C(1-6)alkylidenyl or hydroxy-C(1-3)alkylidenyl; or the aminoalkylidenyl may be a amino-C(1-6)alkylidenyl or amino-C(1-3)alkylidenyl.

The disclosed compounds may include 4-substituted-(4′-hydroxyphenyl)cyclohexane compounds. For example, in the disclosed compounds having Formula I, A-B may be —CH₂CH₂—, A′-B′ may be —CH₂CH₂—, and the compound may have a Formula Ia

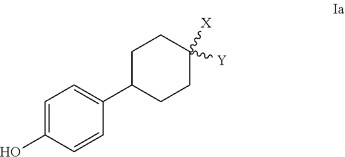

where X and Y are as defined for Formula I. In some embodiments of compounds having Formula Ia, substituent X is selected from hydrogen, hydroxyl, and hydroxyalkyl and Y is selected from hydrogen, hydroxyl, alkyl, and —OCH₂ and Y and Z form a bridge.

The disclosed compounds having Formula Ia may exhibit specific stereochemistry. For example, where X and Y are as defined for Formula I, the compounds may comprise cis and trans isomers of each other. For example, the compounds may comprise cis and trans isomers having the following formula.

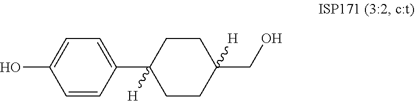 [SMILES: OCC1CCC(c2ccc(O)cc2)CC1]

In particular embodiments, the compound may be the isomer having the formula

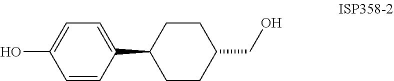 [SMILES: OC[C@H]1CC[C@H](c2ccc(O)cc2)CC1]

In some embodiments of compounds having Formula Ia, X may be hydroxyalkyl and Y may be hydrogen. An exemplary compound may have the formula:

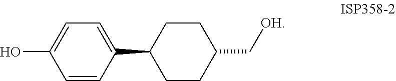 [SMILES: OC[C@H]1CC[C@H](c2ccc(O)cc2)CC1]

In some embodiments of compounds having Formula Ia, X may be hydroxy and Y may be hydrogen. An exemplary compound may have the formula:

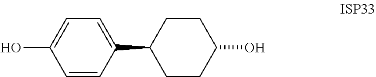 [SMILES: Oc1ccc([C@H]2CC[C@H](O)CC2)cc1]

In some embodiments of compounds having Formula Ia, X may be hydroxyalkyl and Y may be hydroxyl. An exemplary compound may have the formula:

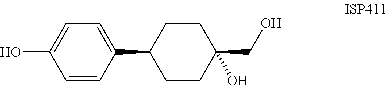 [SMILES: OC[C@]1(O)CC[C@H](c2ccc(O)cc2)CC1]

In some embodiments of compounds having Formula Ia, X may be hydrogen and Y may be —OCH₃— and form a bridge with Z. An exemplary compound may have the formula:

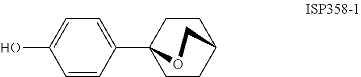

The disclosed compounds may include 4-substituted-(4′-hydroxyphenyl)cyclohexene compounds. For example, in the disclosed compounds having Formula I, A-B may be —CH₂CH₂—, A′-B′ may be ═CHCH₂—, and the compound may have a Formula Ia(i)

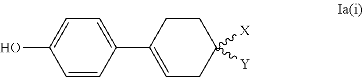

where X and Y are as defined for Formula I. In some embodiments of compounds having Formula Ia(i), substituent X is selected from hydrogen, hydroxyl, and hydroxyalkyl and Y is selected from hydrogen, hydroxyl, alkyl.

In some embodiments of compounds having Formula Ia(i), X may be hydroxyalkyl and Y may be hydrogen. An exemplary compound may have the formula:

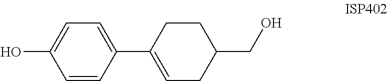 [SMILES: OCC1CC=C(c2ccc(O)cc2)CC1]

The compounds disclosed herein (e.g., compounds having any of Formula I, Ia, and Ia(i) may have several chiral centers, and stereoisomers, epimers, and enantiomers of the disclosed compounds are contemplated. The compounds may be optically pure with respect to one or more chiral centers (e.g., some or all of the chiral centers may be completely in the S configuration; and/or some or all of the chiral centers may be completely in the R configuration; etc.). Additionally or alternatively, one or more of the chiral centers may be present as a mixture of configurations (e.g., a racemic or another mixture of the R configuration and the S configuration). Compositions comprising substantially purified stereoisomers, epimers, or enantiomers of compound having any of Formula I, Ia, and Ia(i) are contemplated herein (e.g., a composition comprising at least about 90%, 95%, or 99% pure stereoisomer, epimer, or enantiomer).

Compositions contemplated herein may include compositions comprising the compound:

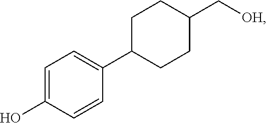 [SMILES: OCC1CCC(c2ccc(O)cc2)CC1]

wherein the isomer

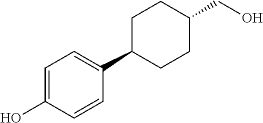 [SMILES: OC[C@H]1CC[C@H](c2ccc(O)cc2)CC1]

of the compound represents the majority of isomers of the compound in the composition (e.g. at least about 90%, 95%, or 99% of the isomers of the compound in the composition).

A compound which is a substantially pure stereoisomer, epimer, or enantiomer is contemplated herein, for example a compound which is at least about 90%, 95%, or 99% pure stereoisomer, epimer, or enantiomer. Contemplated herein is a compound which is a substantially pure (e.g., at least about 90%, 95%, or 99%) stereoisomer, epimer, or enantiomer of the compound:

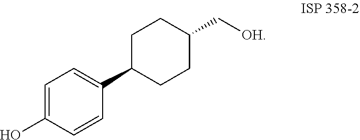 [SMILES: OC[C@H]1CC[C@H](c2ccc(O)cc2)CC1]

Also disclosed herein are hydroxy-protected derivatives of the compounds disclosed herein. For example, the compounds disclosed herein (e.g., compounds having any of Formula I, Ia, and Ia(i), may include a hydroxy-protected group at any hydroxy group. As contemplated herein, a “protected-hydroxy” group is a hydroxy group derivatized or protected by any of the groups commonly used for the temporary or permanent protection of hydroxy functions (e.g., alkoxycarbonyl, acyl, silyl, or alkoxyalkyl groups). A “hydroxy-protecting group” signifies any group commonly used for the temporary protection of hydroxy functions, such as for example, alkoxycarbonyl, acyl, alkylsilyl or alkylarylsilyl groups (hereinafter referred to simply as “silyl” groups), and alkoxyalkyl groups. Alkoxycarbonyl protecting groups are alkyl-O—CO— groupings such as methoxycarbonyl, ethoxycarbonyl, propoxycarbonyl, isopropoxycarbonyl, butoxycarbonyl, isobutoxycarbonyl, tert-butoxycarbonyl, benzyloxycarbonyl or allyloxycarbonyl. As contemplated herein, the word “alkyl” as used in the description or the claims, denotes a straight-chain or branched alkyl radical of 1 to 6 carbons, in all its isomeric forms. “Alkoxy” refers to any alkyl radical which is attached by oxygen (i.e., a group represented by “alkyl-O—”). Alkoxyalkyl protecting groups are groupings such as methoxymethyl, ethoxymethyl, methoxyethoxymethyl, or tetrahydrofuranyl and tetrahydropyranyl. Preferred silyl-protecting groups are trimethylsilyl, triethylsilyl, t-butyldimethylsilyl, dibutylmethylsilyl, diphenylmethylsilyl, phenyldimethylsilyl, diphenyl-t-butylsilyl and analogous alkylated silyl radicals. The term “aryl” specifies a phenyl-, or an alkyl-, nitro- or halo-substituted phenyl group. The terms “hydroxyalkyl”, “deuteroalkyl” and “fluoroalkyl” refer to an alkyl radical substituted by one or more hydroxy, deuterium, or fluoro groups respectively. An “alkylidene” refers to a radical having the general formula —CₖH₂ₖ— where K is an integer (e.g., 1-6). The term “acyl” signifies an alkanoyl group of 1 to 6 carbons, in all of its isomeric forms, or a carboxyalkanoyl group of 1 to 6 carbons, such as an oxalyl, malonyl, succinyl, glutaryl group, or an aromatic acyl group such as benzoyl, or a halo, nitro or alkyl substituted benzoyl group.

The compounds disclosed herein may exhibit binding and agonist and/or antagonist activity for estrogen receptors. As used herein, “ERα” refers to estrogen receptor-alpha, and in particular, human estrogen receptor-alpha. As used herein, “ERβ” refers to estrogen receptor-beta, and in particular human estrogen receptor-beta. Agonists and antagonists for ERα and ERβ are known in the art as are assays for determining the binding affinity of a compound for ERα and ERβ and determining whether a bound compound is an agonist or antagonist for ERα and ERβ. (See e.g., McCullough et al., “Probing the human estrogen receptor-α binding requirements for phenolic mono- and di-hydroxyl compounds: a combined synthesis, binding and docking study,” Biorg. & Med. Chem. (2014) Jan. 1; 22(1):303-10. doi: 10.1016/j.bmc.2013.11.024. Epub (2013) Nov. 21, and the corresponding Supplementary Information, the contents of which are incorporated herein by reference in their entireties). Suitable assays for determining the binding affinity of a compound for ERα and ERβ and determining whether a bound compound is an agonist or antagonist for ERα and ERβ may include fluorescence polarization displacement assays and cell-based ERα and ERβ luminescence activity assays.

As used herein, the term “selective agonist” may be used to refer to compounds that selectively bind and agonize an estrogen receptor, and in particular ERβ, relative to another estrogen receptor, and in particular ERα. For example, a compound that is a selective agonist for ERβ may have an IC₅₀ (nM) in an assay for ERβ receptor agonist activity that is less than 100 nM, preferably less than 10 nM, even more preferably less than 1 nM; and a compound that is that is a selective agonist for ERβ may have an IC₅₀ (nM) in an assay for ERα receptor agonist activity that is greater than 100 nM, preferably greater than 500 nM, even more preferably greater than 1000 nM.

As used herein, the term “selective agonist” may be used to refer to compounds that selectively bind to an estrogen receptor, and in particular, ERβ, relative to another estrogen receptor, and in particular ERα. For example, a compound that is a selective agonist for ERβ may have a binding affinity for ERβ receptor (e.g., as measured by K_{d} (nM)) that is at least 3-fold greater (or at least 5-fold greater, at least 10-fold greater, at least 20-fold greater, at least 50-fold greater, at least 100-fold greater, at least 500-fold greater, or at least 1000-fold greater) than a binding affinity for ERα. Preferably, a selective agonist for ERβ has a K_{d} (nM) for ERβ that is less than 100 nM, more preferably less than 10 nM, or even more preferably less than 1 nM; and preferably, a selective agonist for ERβ has a K_{d} (nM) for ERα that is greater than 500 nM, more preferably greater than 1000 nM, or even more preferably greater than 2000 nM.

As used herein, the term “selective agonist” may be used to refer to compounds that selectively bind and agonize an estrogen receptor, and in particular ERβ, instead of antagonizing an estrogen receptor, and in particular ERβ. For example, a compound that is a selective agonist for ERβ may have an IC₅₀ (nM) in an assay for ERβ receptor agonist activity that is less than 100 nM, preferably less than 10 nM, even more preferably less than 1 nM; and a compound that is that is a selective agonist for ERβ may have an IC₅₀ (nM) in an assay for ERβ receptor antagonist activity that is greater than 100 nM, preferably greater than 500 nM, even more preferably greater than 1000 nM.

Pharmaceutically acceptable salts of the disclosed compounds also are contemplated herein and may be utilized in the disclosed treatment methods. For example, a substituent group of the disclosed compounds may be protonated or deprotonated and may be present together with an anion or cation, respectively, as a pharmaceutically acceptable salt of the compound. The term “pharmaceutically acceptable salt” as used herein, refers to salts of the compounds which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, alpha-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

It should be recognized that the particular counter-ion forming a part of any salt of a compound disclosed herein is usually not of a critical nature, so long as the salt as a whole is pharmacologically acceptable and as long as the counterion does not contribute undesired qualities to the salt as a whole. Undesired qualities may include undesirably solubility or toxicity.

It will be further appreciated that the disclosed compounds can be in equilibrium with various inner salts. For example, inner salts include salts wherein the compound includes a deprotonated substituent group and a protonated substituent group.

The disclosed compounds may be used to prepare and formulate pharmaceutical compositions. As such, also disclosed herein are pharmaceutical compositions comprising an effective amount of any of the compounds disclosed herein, or pharmaceutically acceptable salts of any of the compounds disclosed herein, together with a pharmaceutical excipient. In some embodiments, the disclosed compounds may be used for preparing a medicament for treating a disease or disorder associated with estrogen receptor 13 (ERβ) activity, and in particular, a disease or disorder that may be treated with a specific agonist of ERβ. As such, the disclosed compounds may exhibit ERβ agonist activity, and preferable the compounds exhibit specificity as an ERβ agonist versus an ERβ antagonist, an ERα agonist, and/or an ERα antagonist.

The disclosed compounds may be used to prepare and formulate pharmaceutical compositions for treating diseases that are associated with estrogen ERβ activity. Diseases and disorders associated with ERβ activity may include, but are not limited to, cell proliferative diseases and disorders (e.g., cancers such as breast cancer, ovarian cancer, and endometrial cancer), psychiatric diseases and disorders (e.g., depression, anxiety and/or schizophrenia), neurodegenerative diseases or disorders (e.g., Alzheimer's diseases including APOE4 associated Alzheimer's disease), memory decline (e.g., memory decline observed under low estrogen conditions as those observed in post-menopausal women), bone metabolic diseases or disorders (e.g. osteoporosis), metabolic diseases or disorders (e.g., obesity or insulin resistance), and cardiovascular diseases or disorders. The disclosed pharmaceutical compositions may be administered to subjects in need thereof in methods for treating diseases and disorders associated with ERβ activity.

The compounds and pharmaceutical compositions disclosed herein may be administered to a subject in need thereof to treat a disease or disorder. In some embodiments, the compounds disclosed herein may be administered at an effective concentration such that the compound functions as an agonist for ERβ in order to treat a disease or disorder associated with ERβ activity. In some embodiments, the amount of the disclosed compounds that is effective for the compound to function as an agonist of ERβ is about 0.05-50 μM (or about 0.05-10 μM, or about 0.05-1 μM).

As used herein, a “subject” may be interchangeable with “patient” or “individual” and means an animal, which may be a human or non-human animal, in need of treatment. Suitable subject s for the disclosed methods may include, for example mammals, such as humans, monkeys, dogs, cats, horses, rats, and mice. Suitable human subjects include, for example, those who have a disease or disorder associated with ERβ activity or those who have been determined to be at risk for developing a disease or disorder associated with ERβ activity. A subject in need of treatment may include a post-menopausal woman (e.g., a post-menopausal woman exhibiting low estrogen).

As used herein, a “subject in need of treatment” may include a subject having a disease, disorder, or condition that is responsive to therapy with an ERβ agonist. For example, a “subject in need of treatment” may include a subject having a cell proliferative disease, disorder, or condition such as cancer (e.g., cancers such as breast cancer). In addition, a “subject in need of treatment” may include a subject having a neurological disease or disorder including psychiatric diseases and disorders (e.g., depression, anxiety, and/or schizophrenia). A “subject in need thereof” may include a subject having a neurodegenerative disease or disorder (e.g., Alzheimer's disease including APOE4 associated Alzheimer's disease). In particular, a subject in need thereof may include a subject exhibiting memory loss or the need for enhanced memory consolidation (e.g., a subject having a disease or disorder characterized by a need for enhanced memory consolidation under low-estrogen conditions). A subject in need thereof may include a post-menopausal woman in need of enhanced memory consolidation (e.g., a post-menopausal woman in need of enhanced memory consolidation under low-estrogen conditions).

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with ERβ activity in a subject, whereby the effective amount induces, promotes, or causes ERβ agonist activity in the subject.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

In some embodiments, a daily dose of the disclosed compounds may contain from about 0.01 mg/kg to about 100 mg/kg (such as from about 0.05 mg/kg to about 50 mg/kg and/or from about 0.1 mg/kg to about 25 mg/kg) of each compound used in the present method of treatment. The dose may be administered under any suitable regimen (e.g., weekly, daily, twice daily).

The pharmaceutical compositions for use according to the methods as disclosed herein may include be a single compound as an active ingredient or a combination of compounds as active ingredients. For example, the methods disclosed herein may be practiced using a composition containing a single compound that is an ERβ agonist. Alternatively, the disclosed methods may be practiced using a composition containing two or more compounds that are ERβ agonists, or a compound that is an ERβ agonist together with a compound that is an ERα antagonist.

Instead of administering a pharmaceutical composition comprising a compound that is an ERβ agonist together with a compound that is an ERα antagonist, the disclosed methods may be practiced by administering a first pharmaceutical composition (e.g., a pharmaceutical composition comprising an ERβ agonist) and administering a second pharmaceutical composition (e.g., a pharmaceutical composition comprising an ERα antagonist), where the first composition may be administered before, concurrently with, or after the second composition. As such, the first pharmaceutical composition and the second pharmaceutical composition may be administered concurrently or in any order, irrespective of their names.

As one skilled in the art will also appreciate, the disclosed pharmaceutical compositions can be prepared with materials (e.g., actives excipients, carriers, and diluents etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

The compounds utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. Alternatively, the compounds utilized in the methods disclosed herein may be formulated as a pharmaceutical composition in liquid form (e.g., an injectable liquid or gel)

The compounds utilized in the methods disclosed herein may be formulated as a pharmaceutical composition that includes an excipient, carrier, or diluent. For example, the excipient, carrier, or diluent may be selected from the group consisting of proteins, carbohydrates, sugar, talc, magnesium stearate, cellulose, calcium carbonate, and starch-gelatin paste.

The compounds utilized in the methods disclosed herein also may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents for the pharmaceutical compositions may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

The disclosed pharmaceutical compositions also may include disintegrants. Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

The disclosed pharmaceutical compositions also may include effervescent agents. Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are preferably applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

### Illustrative Embodiments

The following embodiments are illustrative and should not be interpreted to limit the scope of the claimed subject matter.

Embodiment 1. A compound having a formula and stereochemistry as follows:

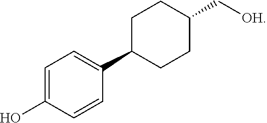 [SMILES: OC[C@H]1CC[C@H](c2ccc(O)cc2)CC1]

Embodiment 2. The compound of embodiment 1 in substantially pure form.

Embodiment 3. A pharmaceutical composition comprising an effective amount of the compound of embodiment 1 preferably in substantially pure form (e.g., where the stereoisomer represents at least about 90%, 95%, or 99% of the compound in the composition), or a pharmaceutically acceptable salt thereof, together with a pharmaceutical excipient, carrier, or diluent.

Embodiment 4. A method for treating a disease or disorder associated with estrogen receptor β (ERβ) activity in a subject in need thereof, the method comprising administering to the subject the compound of embodiment 1 or 2 or the pharmaceutical composition of embodiment 3.

Embodiment 5. The method of embodiment 4, wherein the disease or disorder is selected from neurological, psychiatric, and cell proliferative diseases and disorders.

Embodiment 6. The method of embodiment 4, wherein the disease or disorder is associated with memory loss or memory dysfunction.

Embodiment 7. A method for enhancing memory consolidation in a subject in need thereof, the method comprising administering to the subject the compound of embodiment 1 or 2 or the pharmaceutical composition of embodiment 3.

Embodiment 8. A method for treating a subject exhibiting low estrogen levels, the method comprising administering to the subject the compound of embodiment 1 or 2 or the pharmaceutical composition of embodiment 3.

Embodiment 9. The method of embodiment 7 or embodiment 8, wherein the subject is a post-menopausal woman.

Embodiment 10. A compound having a formula selected from

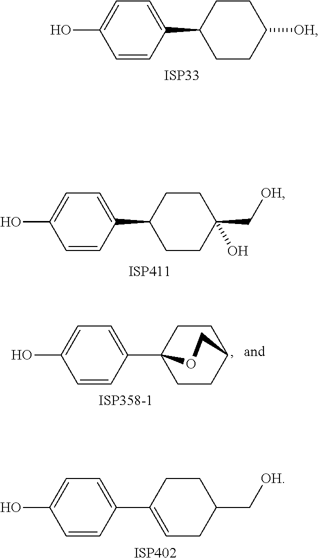

Embodiment 11. A pharmaceutical composition comprising an effective amount of the compound of embodiment 10, or a pharmaceutically acceptable salt thereof, together with a pharmaceutical excipient, carrier, or diluent.

Embodiment 12. A method for treating a disease or disorder associated with estrogen receptor β (ERβ) activity in a subject in need thereof, the method comprising administering to the subject the compound of embodiment 10 or the pharmaceutical composition of embodiment 11.

Embodiment 13. The method of embodiment 12, wherein the disease or disorder is selected from neurological, psychiatric, and cell proliferative diseases and disorders (e.g., cancer such as breast cancer, ovarian cancer, endometrial cancer, and the like).

Embodiment 14. The method of embodiment 12, wherein the disease or disorder is associated with memory loss or memory dysfunction.

Embodiment 15. A method for enhancing memory consolidation in a subject in need thereof, the method comprising administering to the subject the compound of embodiment 10 or the pharmaceutical composition of embodiment 11.

Embodiment 16. A method for treating a subject exhibiting low estrogen levels, the method comprising administering to the subject the compound of embodiment 10 or the pharmaceutical composition of embodiment 11.

Embodiment 17. The method of embodiment 15 or 16, wherein the subject is a post-menopausal woman.

Embodiment 18. A method for enhancing memory consolidation in a subject in need thereof, the method comprising administering to the subject a compound or a pharmaceutical composition comprising the compound having a formula:

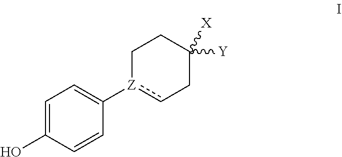

where:
(a) Z is a carbon atom;
(b) X is selected from the group consisting of hydrogen, hydroxyl, alkyl, hydroxyalkyl, amino, and aminoalkyl; and
(c) Y is selected from the group consisting of hydrogen, hydroxyl, alkyl, and hydroxyalkyl; or Y is —CH₂CH₂— or —OCH₂— and Y and Z form a bridge; or X and Y together form alkylidenyl, carboxyalkylidenyl, esteralkylidenyl, hydroxyalkylidenyl, hydroxyalkylalkylidenyl, aminoalkylidenyl, oxo, or oxime.

Embodiment 19. The method of embodiment 18, wherein the compound has a Formula Ia:

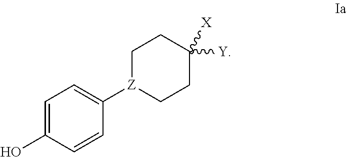

Embodiment 20. The method of embodiment 18 or 19, wherein in the compound X is selected from hydrogen, hydroxyl, alklyl, and hydroxyalkyl; and Y is selected from hydrogen, hydroxyl, alkyl, and hydroxyalkyl; or Y is —OCH₂— and Y and Z form a bridge.

Embodiment 21. The method of embodiment 18, wherein the compound has a Formula Ia(i):

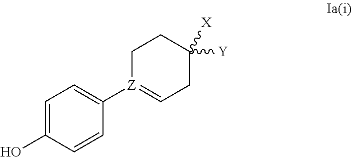

Embodiment 22. The method of embodiment 21, wherein in the compound X is selected from hydrogen, hydroxyl, alkyl, hydroxylalkyl and Y is hydrogen.

Embodiment 23. The method of embodiment 18, wherein in the compound X is hydrogen or methyl, and Y is hydroxymethyl (—CH₂OH) or hydroxyethyl (—CH₂CH₂OH).

Embodiment 24. The method of embodiment 18, wherein in the compound X is methyl, and Y is Y is hydroxymethyl (—CH₂OH).

### EXAMPLES

The following examples are illustrative and should not be interpreted to limit the scope of the claimed subject matter.

### Example 1. A-C Estrogens as Potent and Selective Estrogen Receptor-Beta Agonists (SERBAs) to Enhance Memory Consolidation Under Low-Estrogen Conditions Abstract

Estrogen receptor-beta (ERβ) is a drug target for memory consolidation in post-menopausal women. Herein is reported a series of potent and selective ERβ agonists (SERBAs) with in vivo efficacy that are A-C estrogens, lacking the B and D estrogen rings. The most potent and selective A-C estrogen is selective for activating ER relative to seven other nuclear hormone receptors, with a surprising 750-fold selectivity for the beta over alpha isoform, and with IC₅₀'s of 20-30 nM in cell-based and direct binding assays. Comparison of potency in different assays suggests that the ER isoform selectivity is related to the compound's ability to drive the productive conformational change needed to activate transcription. The compound disclosed herein also shows in vivo efficacy after microinfusion into the dorsal hippocampus, and after intraperitoneal injection (0.5 mg/kg) or oral gavage delivery (5 mg/kg). This simple yet novel A-C estrogen is selective, brain penetrant, and facilitates memory consolidation.

Results

Compound synthesis. Commercially available 4-(4-hydroxyphenyl)cyclohexanone 1 was transformed into 2° or 3° alcohols 2 or 3 by reaction with NaBH₄ or excess methyl lithium respectively, or into oxime 4 by condensation with hydroxylamine (Scheme 1).

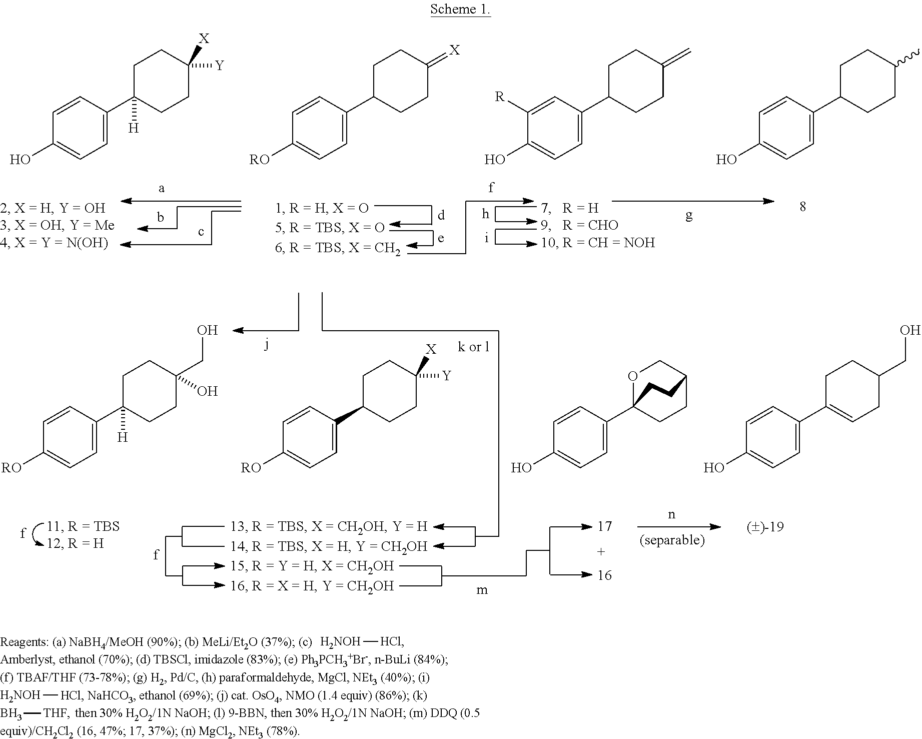

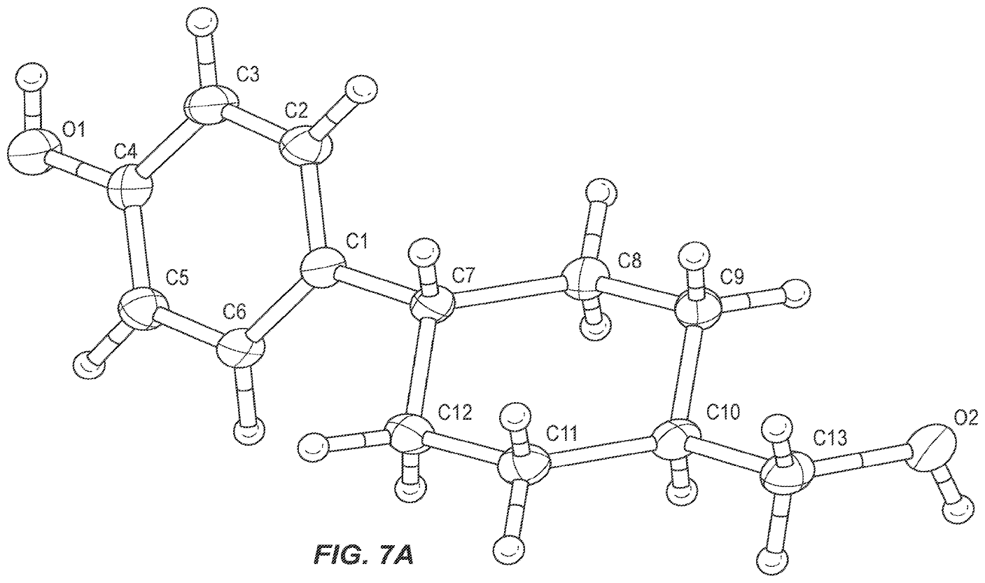

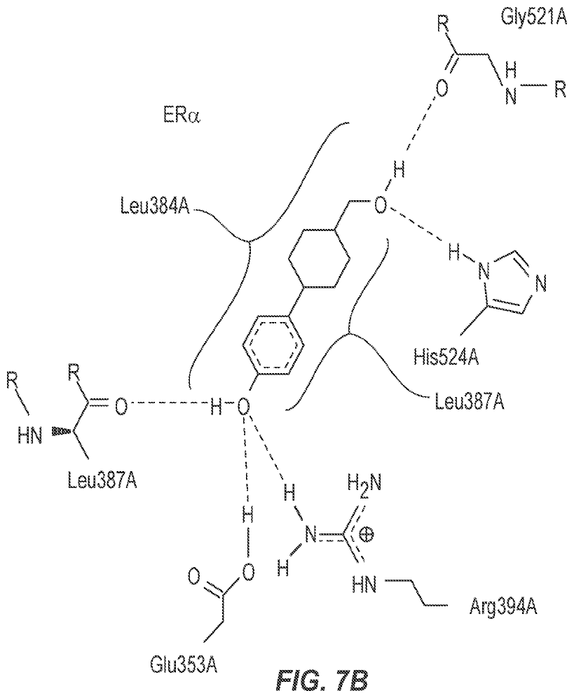

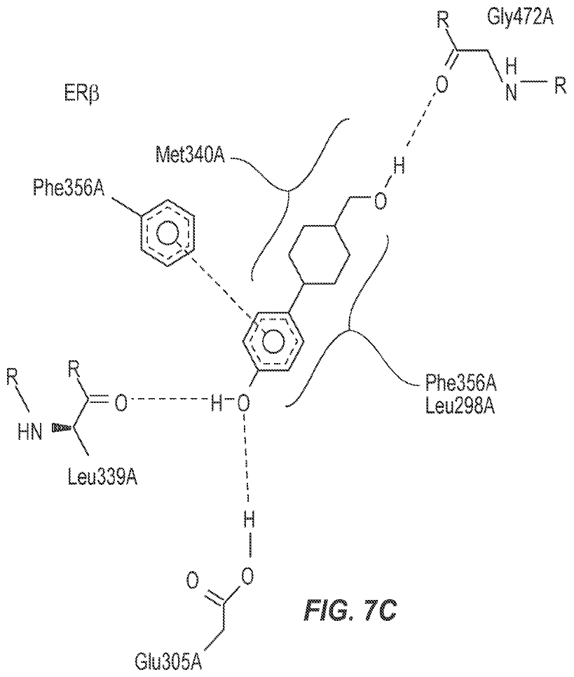

The stereochemistries of 2 and 3 were assigned based on their NMR spectral data. In particular, for 2° alcohol 2, the alcohol methane proton appears as a triplet of triplets at δ 2.38 (J=11.8, 3.4 Hz); the large couplings are consistent with an axial-axial disposition of this proton, and thus the hydroxyl group is equatorial. Signals at δ 69.5 and 31.1 ppm in the ¹³C NMR spectrum of 3, assigned to the 3° alcohol and methyl carbons, are in good agreement with cis-1,4-alcohols of this type²⁴. The t-butyldimethylsilyl ether 5 underwent olefination with the ylide generated from methyltriphenylphosphonium bromide to give 6. Cleavage of the silyl ether using TBAF gave 7; catalytic hydrogenation of 7 gave 8 as a mixture of stereoisomers. Reaction of 7 with excess paraformaldehyde, MgCl₂ and NEt₃ gave the substituted salicaldehyde 9, which upon reaction with hydroxylamine afforded the oxime 10. Dihydroxylation of 6, followed by cleavage of the silyl ether gave 12, as a single stereoisomer after chromatographic purification. The stereochemistry of 12 was assigned as indicated, based on the known stereochemistry of osmium-catalyzed dihydroxylation of 4-t-butylmethylenecyclohexane.²⁵ Hydroboration-oxidation of 6 using BH₃-THF, produced an inseparable mixture of stereoisomeric primary alcohols cis-13 and trans-14, in a 2:1 ratio as determined by integration of the ¹H NMR signals for the hydroxymethylene protons for each (δ 3.60 and 3.39 ppm respectively). The stereochemistry of the isomers was tentatively assigned on the basis of the relative chemical shift of these two signals; the signal for an axial hydroxymethylene (i.e. cis-isomer) appears downfield compared to that for an equatorial hydroxymethylene.²⁴ Alternatively, hydroboration-oxidation using 9-BBN afforded a mixture in which trans-14 was in greater proportion compared to cis-13 (2:3, cis:trans). The use of these two borane reagents to tune the cis:trans outcome for 4-substituted methylenecyclohexanes has previously been reported.^{26,27} Cleavage of the silyl ether using TBAF gave a mixture of stereoisomeric 4-(4-hydroxymethylcyclohexyl)phenols cis-15/trans-16. Treatment of a mixture of the stereoisomers 15/16 (2:3, cis: trans) with DDQ (0.5 equiv.) led to a separable mixture of a bicyclic ether 17 and trans-16. The tentative structural assignment for trans-16 was corroborated by single crystal X-ray diffraction analysis (FIG. 7a).²⁸ Isolation of the unreacted trans-16 is rationalized on the basis of the faster rate of oxidation of cis-15. Since oxidation of either cis- or trans-4-(4-hydroxymethylcyclohexyl)phenol proceeds via the same benzylic carbocation intermediate (i.e. 18, Scheme 2), the activation energy for the formation of this intermediate will be lower for the less stable cis-15 in comparison to trans-16, and thus oxidative cyclization of the cis-isomer will be faster. Reaction of 17 with MgCl₂ and trimethylamine led to an intramolecular elimination reaction to afford the cyclohexene (±)-19 (Scheme 1).

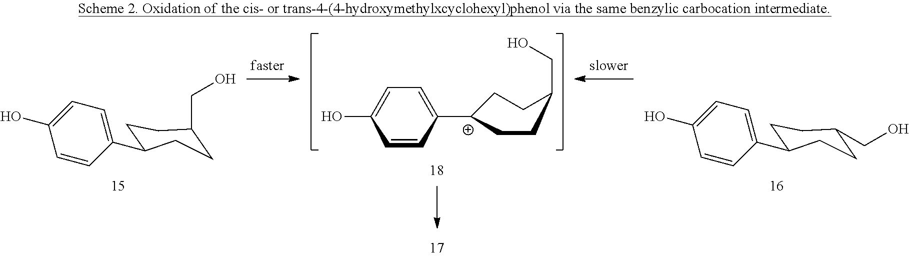

The structure of 19 was assigned on the basis of its NMR spectral data; in particular, the signal for the olefinic proton appears as a narrow multiplet at ca. δ 5.95 ppm. This signal is characteristic of other 1-(4-hydroxyphenyl)cyclohexenes.²⁹

Silyl ether 20 (prepared from 1) underwent Horner-Emmons olefination with triethyl phosphonoacetate to afford the unsaturated ester (±)-21; desilylation with TBAF gave the phenol (±)-22 (Scheme 3).

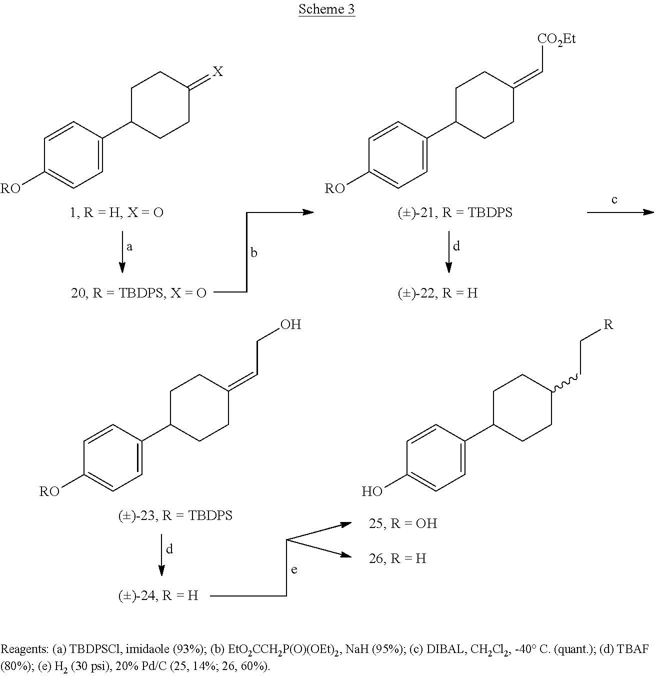

Reduction of 21 with DIBAL, followed by deprotection of the silyl ether yielded the allylic alcohol (±)-24. Catalytic hydrogenation of 24 gave a separable mixture of alcohol 25 and the over reduced ethyl cyclohexane derivative 26.

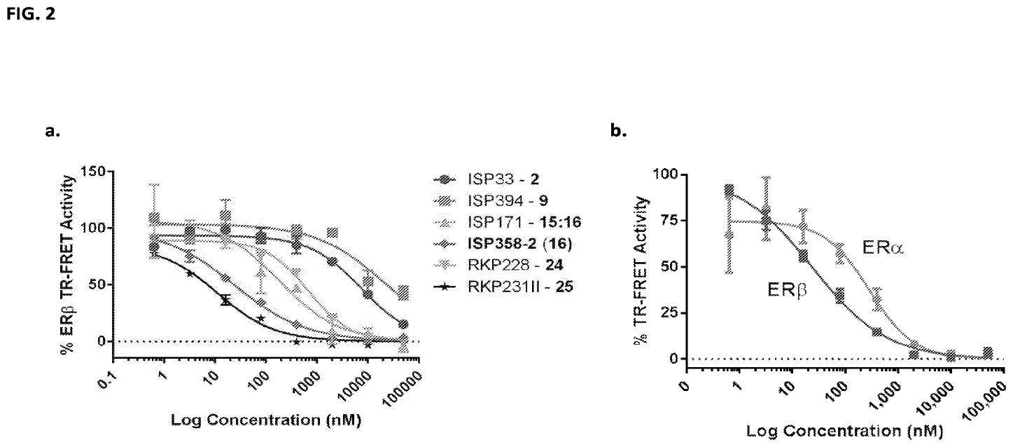

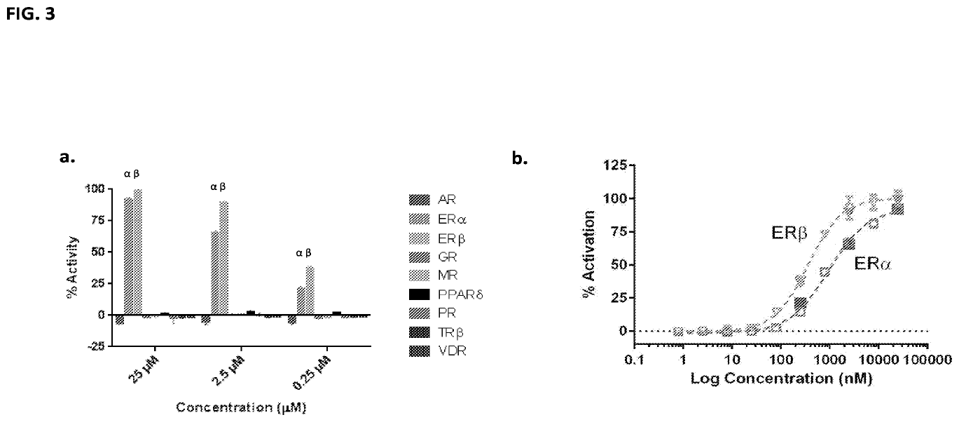

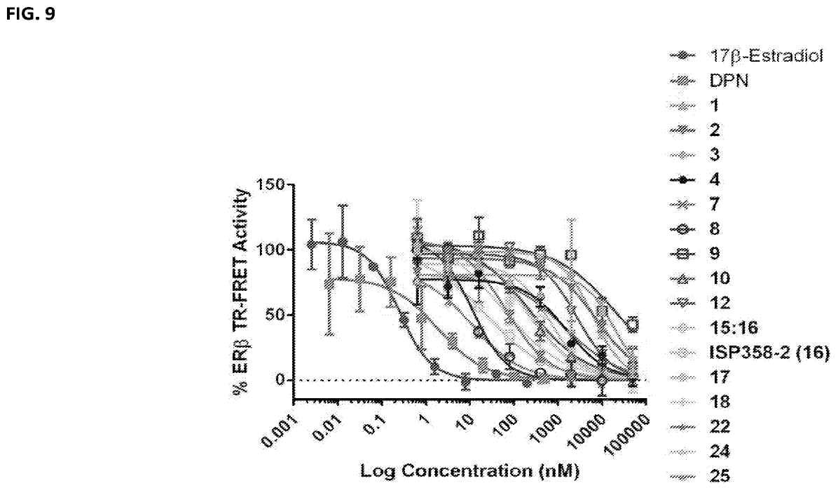

TR-FRET and Cell-based Transcriptional Assays. Initial screening of compounds was performed in a TR-FRET displacement assay, which detects binding to the ERβ LBD (see FIG. 2 for dose-response curves for selected compounds and FIG. 9 for dose-response curves for all compounds). In this assay IC₅₀s were measured for all compounds that were synthesized, with IC₅₀ values summarized in Table 1.

TABLE 1
Estrogen receptor assay data. Reported values are IC₅₀s, with
values in nM.

| | TR- | | | | | |
|---|---|---|---|---|---|---|
| | FRET | ERβ | ERβ | ERα | ERα | ERβ/ERα |
| Compound | ERβ | agon. | antagon. | agon. | antagon. | selectivity |
| Estradiol | 0.25 ± | 0.022 ± | ND | 0.31 ± | ND | 14 |
| | 0.06 | 0.005 | | 0.03 | | |
| DPN | 1.9 ± | 1.1 ± | >10,000 | 2300 ± | >10,000 | 2,090 |
| | 1.3 | 0.12 | | 86 | | |
| 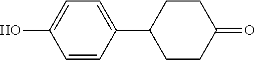 [SMILES: O=C1CCC(c2ccc(O)cc2)CC1] | 19,000 ± 4,300 | ND | ND | ND | ND | — |
| 1 (SM01) | | | | | | |
| 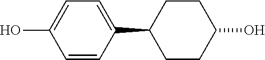 [SMILES: Oc1ccc([C@H]2CC[C@H](O)CC2)cc1] | 7,520 ± 1,304 | ND | ND | ND | ND | — |
| 2 (ISP33) | | | | | | |
| 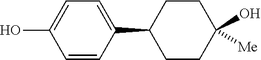 [SMILES: C[C@]1(O)CC[C@@H](c2ccc(O)cc2)CC1] | 6,290 ± 1780 | ND | ND | ND | ND | — |
| 3 (ISP361) | | | | | | |
| 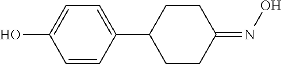 [SMILES: ON=C1CCC(c2ccc(O)cc2)CC1] | 1,570 ± 494 | ND | ND | ND | ND | — |
| 4 (ISP36) | | | | | | |
| 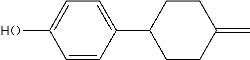 | 66 ± 19 | 101 ± 9 | >10,000 | >10,000 | >10,000 | >99 |
| 7 (ISP365) | | | | | | |
| 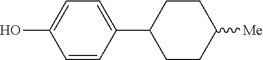 [SMILES: CC1CCC(c2ccc(O)cc2)CC1] | 12 ± 3 | 40 ± 17 | >10,000 | >10,000 | >10,000 | >250 |
| 8 (ISP366) | | | | | | |
| 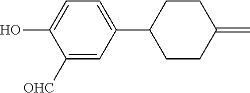 [SMILES: C=C1CCC(c2ccc(O)c(C=O)c2)CC1] | 1,000 ± 430 | ND | ND | ND | ND | — |
| 9 (ISP394) | | | | | | |
| 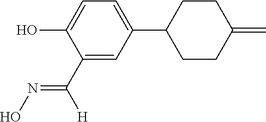 [SMILES: C=C1CCC(c2ccc(O)c(/C=N/O)c2)CC1] | 225 ± 94 | ND | ND | ND | ND | — |
| 10 (ISP389) | | | | | | |
| 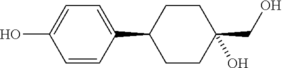 [SMILES: OC[C@]1(O)CC[C@H](c2ccc(O)cc2)CC1] | 2,700 ± 650 | ND | ND | ND | ND | — |
| 12 (ISP411) | | | | | | |
| 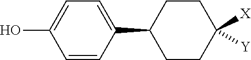 | 1.80 ± 82 | 50 ± 2 | >10,000 | >100,000 | >10,000 | >2,000 |
| 15:16 (3:2) | | | | | | |
| (ISP171) | | | | | | |
| 15, X = CH₂OH, Y = H | | | | | | |
| 16, X = H, Y = CH₂OH | | | | | | |
| 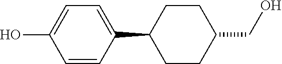 [SMILES: OC[C@H]1CC[C@H](c2ccc(O)cc2)CC1] | 24 ± 5 | 27 ± 4* | >10,000 | 20400 ± 860 | >10,000 | ~750* |
| 16 (ISP358-2) | | | | | | |
| 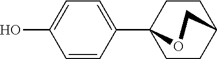 | 250 ± 56 | ND | ND | ND | ND | — |
| 17 (ISP358-1) | | | | | | |
| 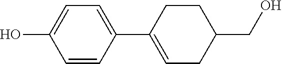 [SMILES: OCC1CC=C(c2ccc(O)cc2)CC1] | 49 ± 29 | 65 ± 13 | >10,000 | >20,000 | >10,000 | >300 |
| (±)-18 (ISP402) | | | | | | |
| 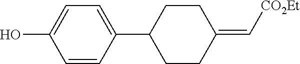 [SMILES: CCOC(=O)C=C1CCC(c2ccc(O)cc2)CC1] | 1,400 ± 480 | ND | ND | ND | ND | — |
| (±)-22 (RKP 230) | | | | | | |
| 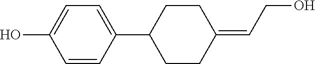 [SMILES: OCC=C1CCC(c2ccc(O)cc2)CC1] | 680 ± 110 | ND | ND | ND | ND | — |
| (±)-24 (RKP288) | | | | | | |
| 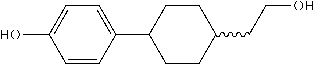 [SMILES: OCCC1CCC(c2ccc(O)cc2)CC1] | 11 ± 3 | 75 ± 19 | >10,000 | >50,000 | >10,000 | >1,000 |
| 25 (RKP231FII) | | | | | | |

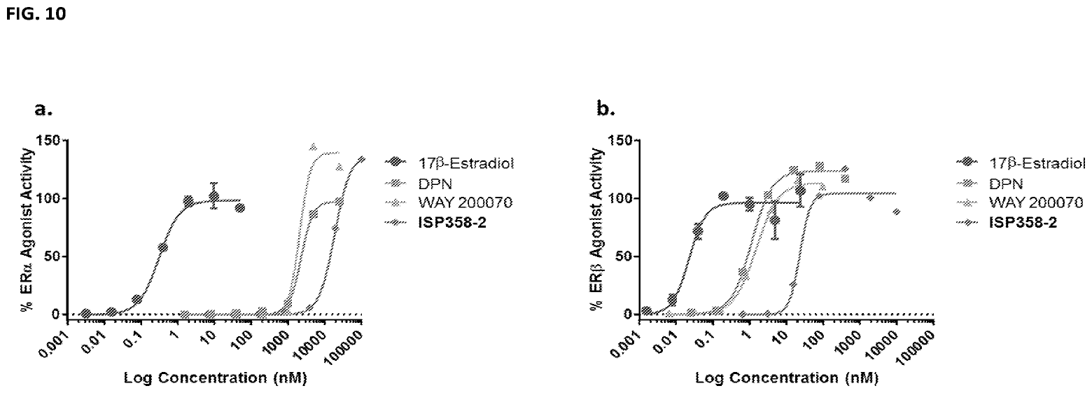

*Average of two data sets with IC₅₀s of 31 ± 7 nM (Figure 5a) and 23 ± 8 nM Figure 10) so selectivity ranges from 658 to 886, with an average of 750.

The most potent compounds were 16 (hereafter referred to as ISP358-2) (hydroxymethyl substitution), 25 (hydroxyethyl substitution), and 8 (methyl substitution) which all had IC₅₀s<30 nM for ERβ. ISP358-2 is the pure trans isomer, and was found to bind with higher affinity to ERβ than the mixture of cis- and trans-stereoisomers (15/16). While having a methylene (ISP358-2) or ethylene (25) linker to the hydroxyl group leads to potency, the direct substitution of the hydroxyl on the cyclohexane ring yields a significant decrease in affinity (IC₅₀ of 7,250 nM for 2). While introduction of unsaturation into the alkyl linker (24) also led to a decrease in affinity (676 nM), introduction of unsaturation into the cyclohexane ring (18) only decreased affinity modestly (49 nM). ISP358-2 was also tested for binding to ERα, and bound with only 12-fold higher affinity to ERβ (IC₅₀ of 24 nM; FIG. 2b) in this TR-FRET assay, which measures direct binding to the isolated LBD.

ISP358-2 was further screened in a nuclear hormone receptor functional assay (FIG. 3), where transcriptional activation was measured due to binding and activation of a chimeric receptor comprised of the LBD of a hormone receptor of interest (e.g. ERβ) tethered to the DNA binding domain (DBD) of GAL4. This assay was done to assess selectivity for activating estrogen receptor, versus other nuclear hormone receptors. No significant agonist activity was observed for compound ISP358-2 for any of the nuclear hormone receptors tested (except for the estrogen receptor) at concentrations ranging from 0.25 to 25 μM (FIG. 3a). Thus, ISP358-2 is not an agonist for the following receptors: Androgen Receptor (AR), Glucocorticoid Receptor (GR), Mineralocorticoid Receptor (MR), Peroxisome Proliferator-Activated Receptor (PPARδ), Progesterone Receptor (PR), Thyroid Hormone Receptor (TRβ), and Vitamin D Receptor (VDR) (see Table 2 for control compound IC50's for each nuclear hormone tested). In a follow-up 10-point titration in this same assay, ISP358-2 was found to be 2.6-fold selective for binding and activating the full-length chimeric ERβ (357+26 nM) relative to full-length chimeric ERα (930±69 nM). This assay (FIG. 3) measures activation of transcription, rather than simply binding of agonist to the ER LBD (as in FIG. 2). But, it uses an unnatural chimeric protein (ER LBD fused to a GAL4 DBD) that may not accurately reflect the actual agonist-induced activation that occurs under native conditions.

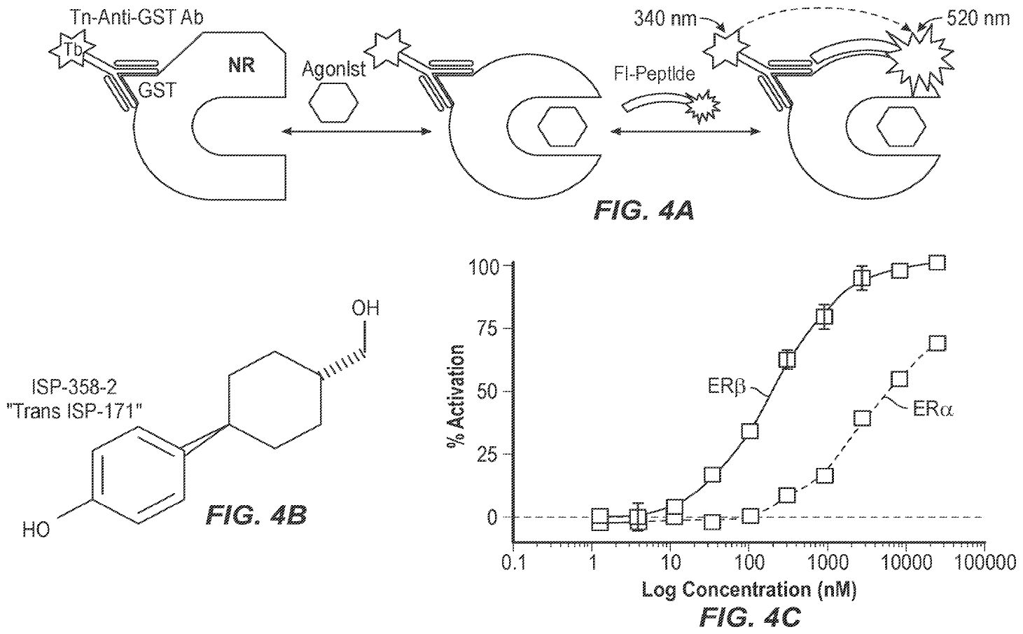

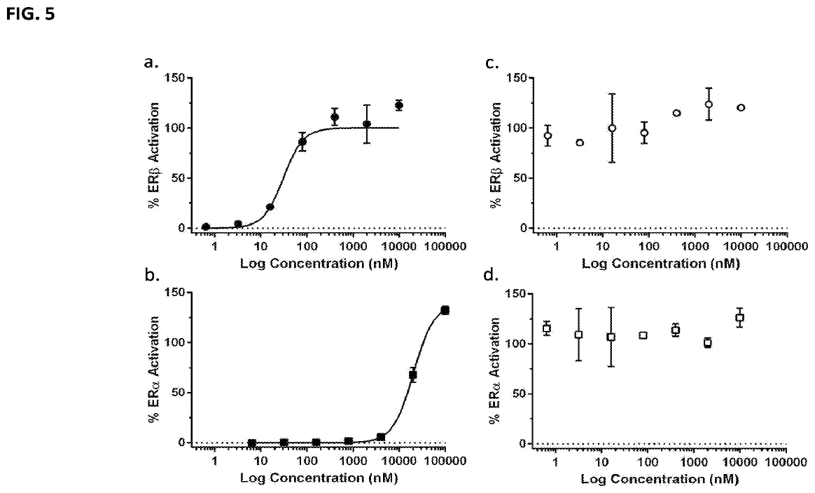

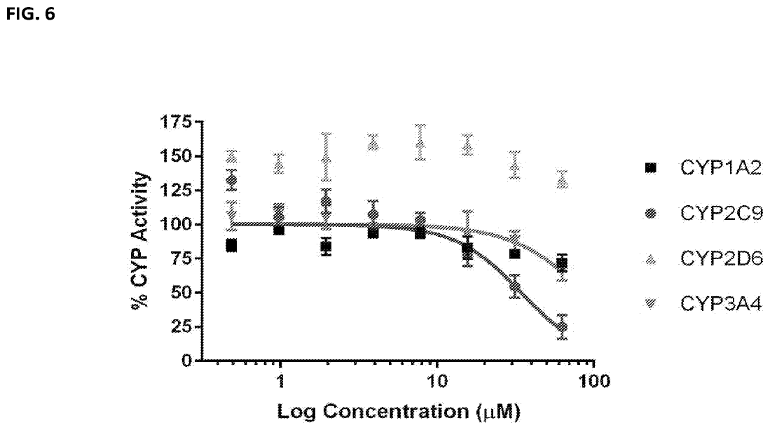

When the coactivator form of the TR-FRET LBD binding assay was performed (FIG. 4a), the ISP358-2 compound (FIG. 4b) was found to be 15-fold selective (FIG. 4c) for binding to ER and recruiting the PPARγ coactivator peptide to ERβ (161±15 nM) relative to ERα (2,940±390 nM). This assay measures activation of the ER LBD, in that it measures binding and agonist-induced recruitment of the coactivator peptide, rather than simply binding of agonist to the receptor.

Finally, a cell-based transcriptional activation assay, which employs a full length and native ER (comprised of an ER LBD and an ER DBD), was performed. Unlike the previous assays, this assay is cell-based, so best mimics the in vivo situation. The most potent and selective compound tested in this assay is ISP358-2, which has an ERβ agonist potency of 31±7 nM (FIG. 5a and FIG. 10a; the assay was performed in duplicate, with values of 31 nM and 23 nM obtained, for an average of 27 nM), and an ERα agonist potency of 20,419+859 nM (FIG. 5b). This makes the ERβ/ERα selectivity ratio ≅750 in this more physiologically relevant assay. ISP358-2 showed no antagonist activity for ERβ (FIG. 5c) or ERα (FIG. 5d) at concentrations up to 10 μM.

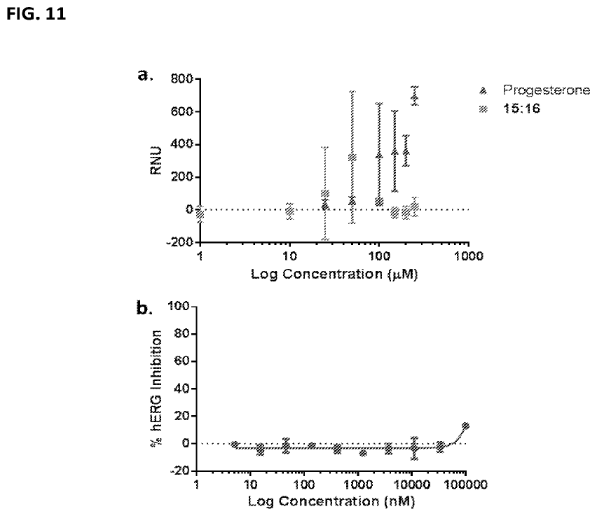

In Vitro Druggability—CYP450 Binding, hERG and Nephelometry. ISP358-2 shows no inhibition of CYP1A2 and CYP2D6, and only weak inhibition of CYP2C9 (IC₅₀=34+4.7 μM) and CYP3A4 (IC₅₀=89±18 μM) (FIG. 6). ISP358-2 does not bind to hERG, showing only 14% activity at 100 μM; and, nephelometry (done for ISP171, 15/16, the mixture of isomers) shows no significant aggregation, indicating good solubility at concentrations up to 300 μM (FIG. 11).

Docking Studies. ISP358-2 (FIG. 7a) was docked into the binding site of agonist-conformation ERα in two conformations with similar docking energy. In one binding mode (FIGS. 6d and 6e), the phenolic hydroxyl interacts with the Arg394/Glu353 (energy=−7.6 kcal/mol), and in another mode the ISP358-2 molecule is flipped 180 degrees (energy=−7.8 kcal/mol) with the aliphatic hydroxyl interacting with Arg394/Glu353. ISP358-2 binds in the agonist-conformation ERβ pocket (FIG. 7c and data not shown) with the phenolic hydroxyl interacting with the Arg394/Glu353 in the lowest energy docking pose (energy=−8.0 kcal/mol). In both cases, there are significant hydrophobic interactions between binding site residues and the bound ISP358-2, although the ERβ pocket is smaller and makes for a tighter fit. In both cases, the hydroxymethyl group is proximal enough (3.0 A) to the His524 to participate in the hydrogen bonding interaction that is typically seen for ER agonists, although in ERβ the hydrogen bond to the aliphatic alcohol may be with the backbone carbonyl of Gly472. In ERβ there are more hydrophobic interactions that constrain the hydroxymethyl-cyclohexyl ring (Ring C) to be nearly planar with the phenolic ring (Ring A) (data not shown), as it is in the native estrogen molecule. This is in contrast to the binding pose in ERα, where the two rings are nearly orthogonal (data not shown). The ERβ hydrophobic interactions are with Phe356, Met340, Phe355, Leu298, near the phenol ring, and with Leu476, Ile373 near the hydroxymethyl group and with Ala302, Leu298 near the cyclohexane ring (FIG. 7c and data not shown). A control docking study of E₂ reproduced the expected binding orientation, based on the crystal structure (data not shown).

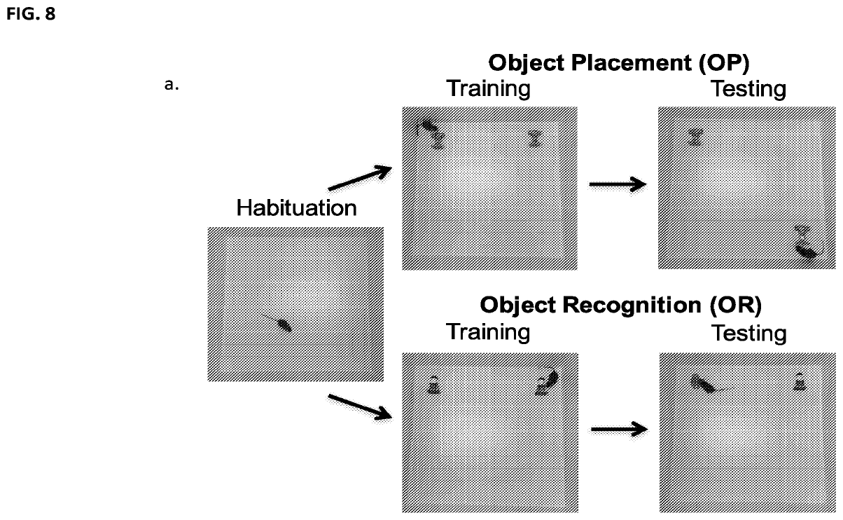

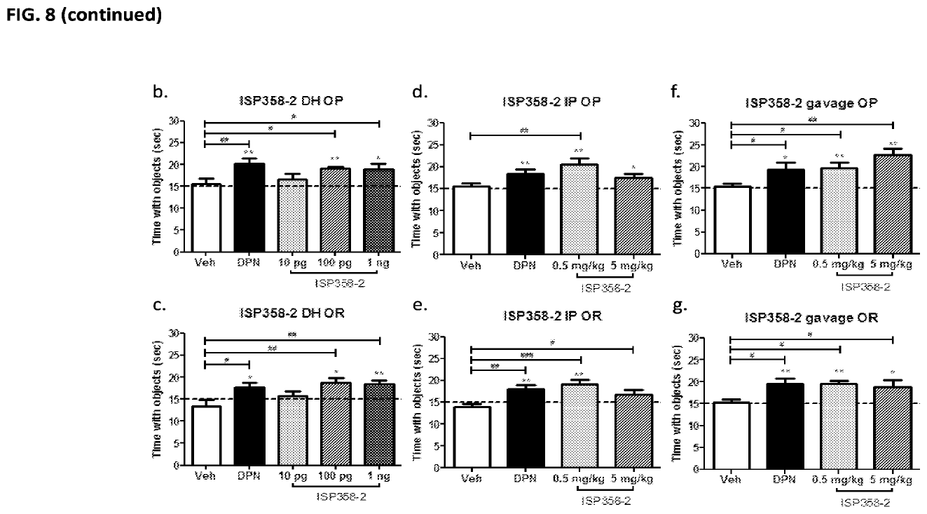

Assessment of Memory Consolidation. Dorsal hippocampal infusion. We first investigated the effects of direct intrahippocampal infusion of ISP358-2 on object recognition and spatial memory consolidation in ovariectomized mice (FIG. 8a). Five groups of mice were tested (FIG. 8b,c): vehicle (negative control), DPN (positive control), and three doses of ISP358-2 (10 pg/hemisphere, 100 pg/hemisphere, and 1 ng/hemisphere). For object placement (FIG. 8b), one-sample t-tests indicated that mice receiving vehicle or 10 pg ISP358-2 did not spend significantly more time than chance with the moved object (ts₍₇₎=0.44 and 1.19, respectively, p>0.05; n=8), indicating that these groups did not exhibit a memory for the training object location. In contrast, mice receiving DPN, 100 pg ISP358-2, or 1 ng ISP358-2 spent significantly more time than chance with the moved object (ts₍₆₎=4.5, 10.3, and 3.4, respectively, p<0.05; n=7), indicating robust memories for the training object location. In addition, a one-way ANOVA conducted on the time spent with the moved object indicated a significant main effect of treatment (F_{(4,32)}=2.97, p=0.034). Fisher's LSD posthoc tests indicated that the DPN, 100 pg, and 1 ng groups spent significantly more time with the moved object than the vehicle group, whereas the vehicle and 10 pg groups did not differ from each other. Together, these data suggest that dorsal hippocampal infusion of 100 pg or 1 ng ISP358-2 enhanced object placement memory consolidation.

Results for object recognition (FIG. 8c) were nearly identical. Neither the vehicle nor 10 pg ISP358-2 groups showed a preference for the novel object (ts₍₉₋₁₀₎=1.08 and 0.88, respectively, p>0.05; n=10-11). However, the DPN, 100 pg ISP358-2, and 1 ng ISP358-2 groups all spent significantly more time than chance with the novel object (ts₍₉₋₁₀₎=2.35, 3.16, and 1.08, respectively, p<0.05; n=10-11). Moreover, the main effect of treatment was significant (F_{(4,48)}=3.69, p=0.011), and posthoc tests confirmed that the DPN, 100 pg ISP358-2, and 1 ng ISP358-2 groups, but not the 10 pg ISP358-2 group, differed significantly from vehicle. As with object placement, these data indicate that dorsal hippocampal infusion of 100 pg or 1 ng ISP358-2, but not 10 pg ISP358-2, enhanced object recognition memory consolidation.

Intraperitoneal injection. We next used a new set of mice to investigate whether systemic administration of ISP358-2 also provide similar memory enhancing effects as intrahippocampal infusion (FIG. 8d,e). Intraperitoneal (IP) injection is a common, reliable, and convenient systemic treatment in which the injected drug is absorbed into the blood vessels through the peritoneum.³⁰ Because the doses of the drugs for intrahippocampal infusion are much smaller than that needed to cross the blood brain barrier, we examined a range of IP doses based on the cell-based assay and DH infusion results above and previous work showing that IP injections of 0.05 mg/kg DPN enhanced object recognition memory.³¹ In our cell-based assays, the IC50 of ISP358-2 was approximately 10 times higher than that of DPN. Moreover, our behavioral tests showed that ISP358-2 enhances hippocampal memory at a concentration 10 times higher than DPN. Therefore, our IP doses of ISP358-2 were at least 10 times higher than DPN (0.5 mg/kg and 5 mg/kg). We thus tested four groups of mice as follows: vehicle (negative control), DPN (positive control), and two doses of ISP358-2 (0.5 mg/kg and 5 mg/kg).

For object placement (FIG. 8d), one-sample t-tests indicated that mice receiving vehicle did not show a preference for the moved object (t₍₈₎=0.68, p>0.05; n=9). However, the DPN, 0.5 mg/kg ISP358-2, and 5 mg/kg ISP358-2 groups all spent significantly more time than chance with the moved object (ts₍₉₋₁₁₎=3.20, 3.93, and 2.78, respectively, p<0.05; n=10-12), suggesting that systemic administration of ISP358-2 enhanced object placement memory consolidation. Moreover, the main effect of treatment was significant (F_{(3,38)}=3.63, p=0.021), and posthoc tests showed that the 0.5 mg/kg ISP358-2 group differed significantly from vehicle. These data demonstrate that IP injection of ISP358-2 enhances spatial memory consolidation in a manner similar to dorsal hippocampal infusion.

Similar results were observed for object recognition (FIG. 8e). One-sample t-test results showed that mice receiving vehicle did not spend significantly more time than chance with the novel object (t₍₉₎=1.40, p>0.05; n=10). In contrast, the DPN and 0.5 mg/kg ISP358-2 groups exhibited a significant preference for the novel object relative to chance (ts_{(8 and 11)}=3.52 and 4.17, respectively, p<0.01; n=12 and 9). There was also somewhat of a trend for the 5 mg/kg ISP358-2 to prefer the novel object (412)=1.65, p=0.125; n=13). In addition, the main effect of treatment was significant (F_{(3,40)}=5.05, p=0.005), and posthoc tests verified that the DPN, 0.5 mg/kg ISP358-2, and 5 mg/kg ISP358-2 groups differed significantly from the vehicle group. Together, the object placement and object recognition data suggest that IP administration of ISP358-2, particularly the 0.5 mg/kg dose, enhances object recognition and spatial memory consolidation similar to dorsal hippocampal infusion. Importantly, these data also demonstrate brain penetrance and behavioral efficacy in ovariectomized mice.

Oral gavage. Given the mnemonic effectiveness of IP injection, we next assessed whether oral administration of ISP358-2 could enhance memory consolidation (FIG. 8f,g). Oral gavage is a common procedure in scientific experiments delivering the drug directly into the stomach by means of a syringe.³² Although it is highly effective and more accurate than other oral administration methods such as administration through delivery in food and/or water, it is more invasive and stressful.³³ Because we observed that IP injection of ISP358-2 enhanced hippocampal memory consolidation, we used the same doses for oral gavage as for IP injections (vehicle, 0.5 or 5 mg/kg ISP358-2 and 0.05 mg/kg DPN).

Similar results were observed for oral administration as were observed for IP injection ISP358-2. For object placement (FIG. 8f), one-sample t-tests results showed that mice receiving vehicle did not spend significantly more time than chance with the moved object (t₍₈₎=0.54, p>0.05; n=9). However, the DPN, 0.5 mg/kg ISP358-2, and 5 mg/kg ISP358-2 groups all exhibited a significant preference for the moved object relative to chance (ts₍₈₋₉₎=2.76, 3.65, 5.06, respectively, p<0.05; n=9-10), suggesting that oral administration of ISP358-2 enhanced spatial memory consolidation. Also, one-way ANOVA showed that the main effect of treatment was significant (F_{(3,33)}=5.04, p=0.006), and posthoc tests verified that the DPN, 0.5 mg/kg ISP358-2, and 5 mg/kg ISP358-2 groups differed significantly from the vehicle group. Likewise, for object recognition (FIG. 8g), one-sample t-tests indicated that mice receiving vehicle did not show a preference for the novel object (t₍₈₎=0.25, p>0.05; n=9). In contrast, the DPN, 0.5 mg/kg ISP358-2, and 5 mg/kg ISP358-2 groups all spent significantly more time with the novel object relative to chance (ts₍₇₋₉₎=3.89, 5.37, 2.36, respectively, p<0.05; n=8-10). Moreover, the main effect of treatment was significant (F_{(3,32)}=3.02, p=0.044), and posthoc tests showed that the DPN, 0.5 mg/kg ISP358-2, and 5 mg/kg ISP358-2 groups differed significantly from vehicle. Together, the object placement and object recognition behavior results demonstrate that oral administration of ISP358-2 enhances object recognition and spatial memory consolidation similar to dorsal hippocampal infusion or IP injection. These data also suggest the oral bioavailability of ISP358-2 in ovariectomized mice.

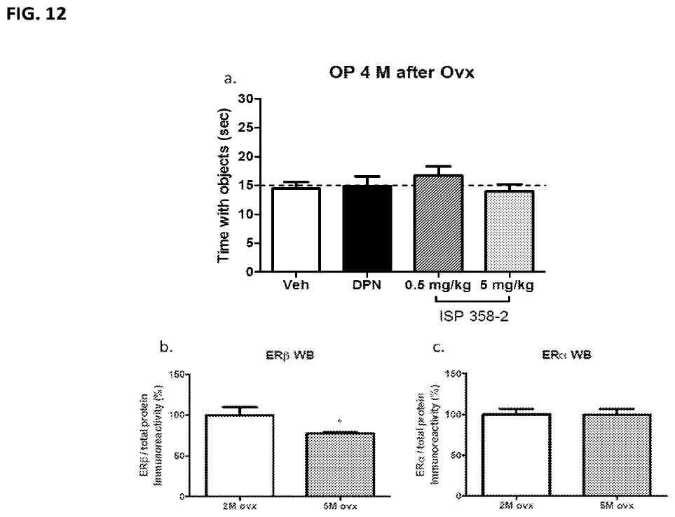

Finally, we collected preliminary data to assess the effectiveness of orally-gavaged ISP358-2 on spatial memory consolidation in mice who experienced long-term estrogen deprivation. Mice that received i.p. injections of vehicle, DPN, or ISP358-2 above remained in our colony for 4 months after ovariectomy. They were then trained in the object placement task (using new objects) and then immediately given vehicle, DPN, or ISP358-2 via oral gavage in the same doses described above (n=9-12/group). Unlike mice gavaged within 1 month of ovariectomy (FIG. 8f), DPN or ISP358-2 did not enhance spatial memory consolidation in mice treated within 4 months of ovariectomy (FIG. 12a). We then measured ERα and ERβ levels in the DH using Western blotting as per our previous work³⁴ (ERα, 1:200, Santa Cruz Biotechnology; ERβ, 1:200, Santa Cruz Biotechnology); tissue was collected approximately 2 and 5 months after ovariectomy. Levels of ERβ were significantly reduced 5 months after ovariectomy relative to 2 months after ovariectomy (FIG. 12b; t₉=2.46, p<0.05). In contrast, levels of ERα did not change (FIG. 12c). These data suggest that DPN or ISP358-2 did not enhance memory after long-term ovariectomy, because of the reduced levels of ERβ. By extension, these data also support in vivo selectivity of ISP358-2 for ERβ; because, if ISP358-2 enhanced memory via binding to ERα, then it should have been able to enhance memory consolidation after long-term ovariectomy because ERα levels were not reduced. However, the fact that ERβ levels were reduced at a time at which ISP358-2 did not enhance memory supports our hypothesis that that it regulates memory via ERβ and not ERα.

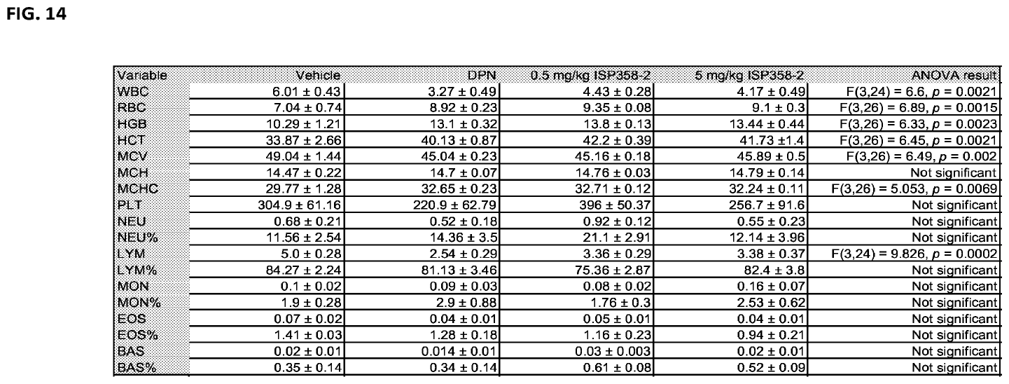

Assessment of Peripheral Pathology or Cell Proliferation Due to ISP358-2 Treatment. In general, all the tissues from the twenty different specimens appeared similar. Heart: the cardiac tissues were all unremarkable. The ventricular walls were intact and normal thickness. The atrial walls were intact with normal thickness. There was no evidence of congenital defects such as myofiber disarray or ischemic heart disease or ischemic injury. There was no evidence of inflammation or myocarditis. Kidney: The kidneys were all unremarkable. The glomeruli were intact. The tubules appeared normal. There was no evidence of inflammation involving any of the structures of the kidneys. Liver: The general architecture of the liver was intact and normal appearing with large portal-types veins running together with hepatic ducts and hepatic arteries. The central veins were present and normal appearing. A generalized appearance of low grade/mild ischemic injury was present in all samples. This seemed non-specific, was appreciated in all specimens, and could be secondary to early ischemic damage or autolysis that occurred post mortem. In several animals, small foci of cellular necrosis were observed likely secondary to ischemia. Two animals showed areas with mild, low grade inflammation that was small and focal. One animal (R15-IP-24 V) had multifocal areas of an organized inflammatory infiltrate composed primarily of mononuclear lymphocytes. Overall there was no evidence of acute inflammation composed of neutrophils or damage to structures in the liver, such as hepatic ducts. Bloodwork chemistry and hematology data (FIG. 14) for treated animals do not show significant deviations from expected reference ranges, relative to vehicle, except for modest effects due to hemolysis that was likely due to sample collection via cardia puncture. Finally, while E₂ caused statistically significant proliferation of MCF-7 breast cancer cells at doses of 10, 100 and 1,000 μM, neither ISP358-2 nor DPN showed any significant proliferation, relative to untreated control cells.

### Discussion

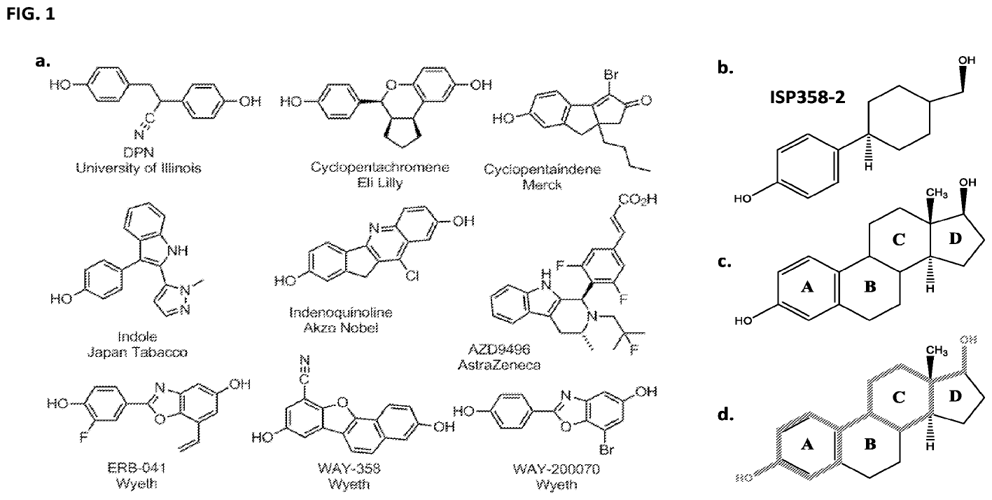

ERβ has previously been pursued as a drug target for a wide range of conditions, including anxiety, depression, schizophrenia, and Alzheimer's disease, with representative ERβ drug lead agonist compounds shown in FIG. 1a.³⁵ Compounds presented herein (Table 1) differ from these previously reported compounds in that they are more selective for ERβ over ERα (≅750 fold), and in that they are A-C estrogens that resemble the native 17β-estradiol molecule, lacking only the B and D rings.

Structure Activity Relationship for the A-C Estrogens. The binding affinity of 4-(4-substituted cyclohexyl)phenols were assessed in a TR-FRET ERβ binding assay (Table 1 and FIG. 2). In particular, compounds bearing a hydroxymethyl functionality attached to the cyclohexyl core showed higher affinities, in the range 20-200 nM. Of the two components in the 2:1 mixture of cis- and trans-stereoisomers 15/16 (IC₅₀=184 nM), the trans-isomer was found to be more potent (ISP358-2, IC₅₀=24 nM) than the mixture. Introduction of unsaturation within the six-membered ring (18, IC₅₀=49 nM) did not greatly reduce the binding affinity compared to ISP358-2; however, conformational restriction, such as is present in the exocyclic allylic alcohol, reduced affinity (24, IC₅₀=676 nM). The presence of a third hydroxyl group led to greatly reduced binding affinity (12, IC₅₀=2,700 nM). Finally, varying the distance between the phenolic OH and the aliphatic alcohol group gave a larger range of binding affinities (2, IC₅₀=7250 nM, 25, IC₅₀=11 nM). Those analogs having IC50 values <200 nM were further tested in a cell-based transcriptional activation assay to evaluate their ERβ selectivity both in terms of binding affinity and of efficacy in activating transcription, in a biologically relevant system. The trans-stereoisomer ISP358-2, 8, 18, and 25 exhibited similar ERβ agonist potencies (EC₅₀˜30-75 nM) in the TR-FRET assay; but, ISP358-2 stands out as being the most potent and selective in this more biologically relevant cell-based functional assay. Interestingly, the hydroxyethyl analog was less potent in the cell-based functional assay (25, EC₅₀=75 nM vs. 11 nM).

The potency differences observed in the assays may be due to the nature of what the assays measure. The TR-FRET assay in FIG. 2 measures only displacement of a fluorescently labelled estradiol ligand from the ligand binding domain (LBD), which reflects binding affinity for the ligand that competitively displaces the fluorescent probe. In contrast, the cell-based assay is more complicated and measures the entire sequence of molecular events leading to transcriptional activation. This sequence of events involves a series of conformational changes (e.g. a rotation of Helix-12 of the ligand binding domain) that are triggered by the initial hormone binding.³⁶ The protein conformational change in the estrogen receptor induced by agonist binding results in recruitment of a coactivator protein and also triggers protein dimerization, ultimately resulting in DNA binding and transcriptional activation. Additional interactions between the aliphatic hydroxyl group and the His475 residue of ERβ plays a role in the conformational change involving nearby Helix-12, which would be reflected in IC₅₀ values in the cell-based functional assay (FIG. 5); but, not in the TR-FRET binding assay (FIG. 2).

All compounds tested showed no significant ERβ or ERα antagonist activity (EC₅₀>10,000 nM), thus also demonstrating their selectivity as agonist vs. antagonist activity (Table 1 and FIG. 5c,d). Of the ERβ agonists, ISP358-2 was the most selective, with ≅750-fold agonist selectivity for ERβ over ERα in the cell-based functional assay. But, it had only modest selectivity in the TR-FRET binding assay (FIG. 2b).

Assay Differences Suggest Mechanism for Isoform Selectivity. As mentioned above, the cell-based assay for ISP358-2 indicates that it is ≅750 fold selective for ERβ agonist activity over ERα agonist activity (FIG. 5a,b); whereas, the TR-FRET binding assay shows more modest 12-fold selectivity for ERβ (FIG. 2b). This could be because the TR-FRET binding assay simply measures binding affinity (to an isolated LBD), whereas the cell-based assay measures transcription that is induced by agonist binding to a full-length and native ERβ, which causes a productive conformational change in ERβ that includes rotation of Helix-12 (leading to recruitment of coactivators, dimerization, DNA binding and then transcriptional activation). To test the hypothesis that assay differences are due to these downstream activation events, two other assays were performed. In a first assay, transcriptional activation was measured in a different cell-based assay, now using an unnatural chimeric receptor (ER LBD fused to a GLA4 DBD). In this assay (FIG. 3b), there was a modest 2.6-fold selectivity for ERβ. In a second assay, ability of agonist binding to recruit binding of a coactivator peptide to the ER LBD was measured (FIG. 4a). In this assay, 15-fold selectivity for ERβ was observed (FIG. 4c). Thus, ERβ versus ERα selectivity for ISP358-2 varies significantly, based on how well the assay incorporates native downstream activation events that occur subsequent to binding to the ERβ binding pocket, as a result of hormone-induced conformational changes. It therefore appears that the significant ERβ versus ERα selectivity shown by ISP358-2 is not just a function of binding affinity for the ERβ receptor (as measured in the TR-FRET assay in FIG. 2b); but, rather is a function of the ability to induce the productive conformational change that leads to downstream activation of transcription (FIG. 5a).

Consistent with the above hypothesis that ISP358-2 potency and selectivity is related to its ability to drive a productive conformational change, docking studies show that ISP358-2 docks into the ERβ active site in a conformation that differs significantly from that for the ERα binding site. Key differences occur where the estrogen C ring is normally located (FIG. 7c and data not shown), thereby affecting positioning of the aliphatic hydroxyl group that interacts with the His524 and/or Gly472 (backbone carbonyl) residues (FIG. 7c and data not shown) in the region known to be important for driving the Helix-12 conformational change that permits binding of coactivator. The ERβ hydrophobic interactions include Pi-Pi stacking between Phe356 and the ISP358-2 phenol ring, along with Ala302, Leu298, Leu476 and Ile373 that constrain the cyclohexyl ring and its attached hydroxymethyl methyl group in the “C ring” region (FIG. 1d). These unique hydrophobic interactions in the ERβ active site may be what drives the 90° rotation of the C (cyclohexane) ring of ISP358-2 relative to the phenol ring, in the ERβ pocket relative to the ERα pocket (FIG. 7c and data not shown); and, in this way they could affect the adjacent coactivator pocket. This region of the 17β-estradiol binding pocket is known to affect the accessibility and structure of the coactivator binding pocket, so could be the reason for the large differences in agonist activity that were observed for ISP358-2. Future structural characterization studies are being planned to address this question.

Druggability and Preliminary Safety Toxicity. While ISP358-2 binds to ER and activates transcription, it shows no significant off-target activity with seven other nuclear hormone receptors (FIG. 3a). ISP358-2 also showed no significant activity against the heart potassium ion channel hERG (FIG. 11b) and no significant inhibition of the major drug metabolizing cytochrome P450 enzymes, CYP2D6, CYP3A4, CYP2C9 and CYP1A2 (FIG. 6). ISP358-2 was also reasonably soluble, showing no aggregation in nephelometry assays (FIG. 11a).

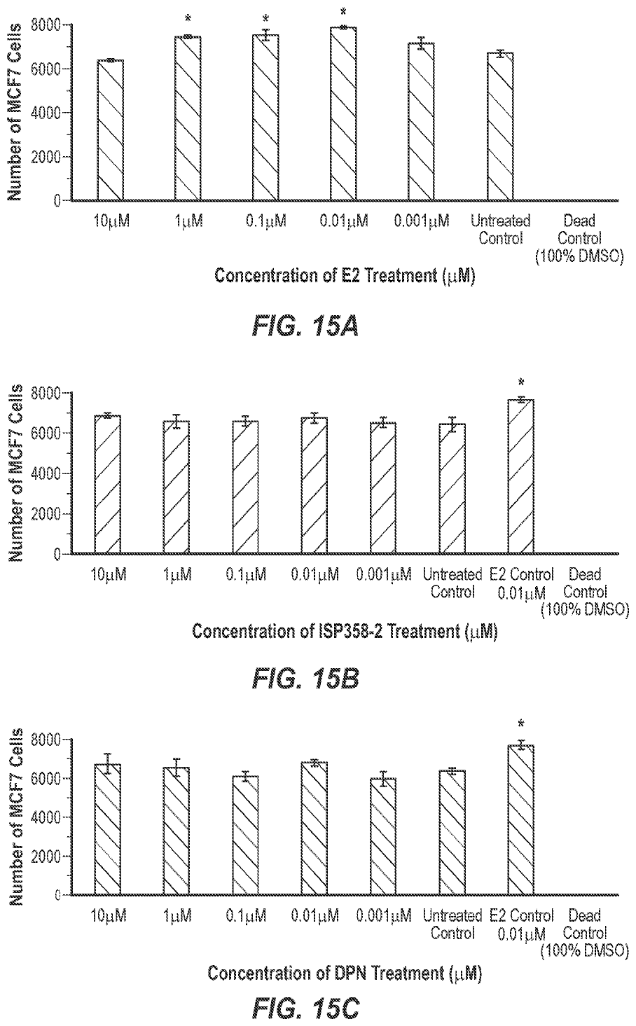

To assess the potential of ISP358-2 to stimulate breast cancer cell growth, MTT assays with MCF-7 human breast cancer cells were performed (FIG. 15). No significant changes in the growth of MCF-7 cells were observed following treatment at any concentration of the ERβ agonists ISP358-2 or DPN compared to untreated controls (FIG. 15b,c). However, the proliferation of MCF-7 cells treated with 1, 0.1 or 0.01 μM E₂ was significantly increased (n=3; p<0.02, 0.05, 0.00, respectively) compared to untreated controls (FIG. 15a). Furthermore, cell proliferation was significantly lower (n=3; p<0.04 for both compounds) in comparison to positive control MCF-7 cells treated with 0.01 μM E₂.

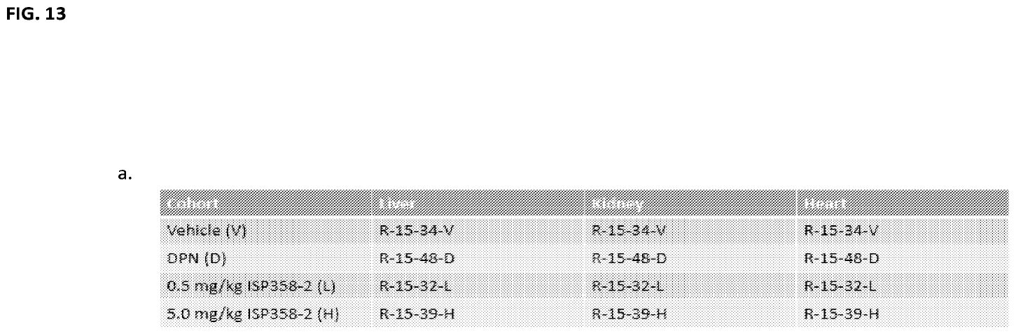

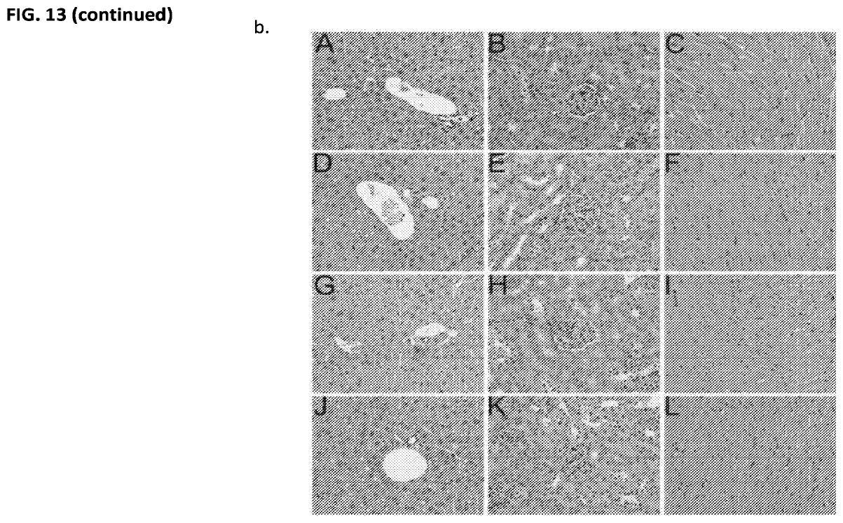

To assess potential peripheral pathology due to ISP358-2 treatment, a histological analysis of tissue slices of treated animals was performed (FIG. 13). Overall, tissue changes due to treatment were unremarkable. Mild, global ischemic changes were noted in the livers of all animals. It is difficult to assign a specific pattern or significance to this finding. These changes likely represent hypo-perfusion and subsequent mild ischemic changes in the post mortem period. One animal showed organized lymphoid hyperplasia in the liver. None of the animals demonstrated any significant pathological changes in the heart or kidney.

In vivo Efficacy. In vivo behavioral assays, measuring object placement or object recognition (FIG. 8a), showed efficacy for all three routes of administration: microinfusion into the dorsal hippocampus, intraperitoneal injection, or oral gavage (FIG. 8). Thus, ISP358-2 can enhance object recognition and spatial memory consolidation in ovariectomized female mice. Intrahippocampal infusion of 100 pg and 1 ng ISP358-2 enhanced memory consolidation in the object recognition and object placement tasks as effectively as the ERβ agonists DPN (FIG. 8b,c). In systemic administration experiments, 0.5 mg/kg ISP358-2 most effectively enhanced consolidation in both tasks when delivered intraperitoneally (FIG. 8d,e), whereas 5 mg/kg ISP358-2 was most effective via oral gavage (FIG. 8f,g). These data are consistent with previous findings showing that intrahippocampal or systemic administration of the ERβ agonists DPN or WAY200070 enhance hippocampal-dependent memory in ovariectomized rats and mice in tasks including object recognition, object placement, and the radial arm maze.^{3, 34, 39-42} As such, ISP358-2 mimics the memory-enhancing effects of other ERβ agonists with different chemical structures and could potentially be used to reduce memory dysfunction in numerous neuropsychiatric conditions for which women are at increased risk, including AD, depression, and schizophrenia.⁴³ Moreover, women are at greater risk of anxiety disorders than men,⁴³ and DPN decreases anxiety-related behaviors among rodents tested in the open field and elevated plus maze tasks.^{44, 45} Thus, ISP358-2 has the potential to not only facilitate memory consolidation but also to reduce anxiety. Although promising, numerous issues remain to be addressed in future studies, including the extent to which the beneficial effects of ISP358-2 generalize to males, older subjects, rodent models of AD and other disorders, and other forms of memory.

Finally, while it was observed that ISP358-2 is highly selective for ERβ over ERα in the more biologically relevant cell-based assay, it is not known if it has this same selectivity for ERβ in vivo. However, our preliminary studies have shown a correlation between behavioral results and levels of ERβ in the brain, consistent with the effect being related to ERβ agonist activity (FIG. 12). Future studies will be directed to determining the pharmacological mechanism of ISP358-2 in vivo, including studies of isoform selectivity, effects on signaling cascades and neural morphology changes in the brain, as well as pharmacokinetics and pharmacodynamics.

### CONCLUSION

The results of the current study demonstrate that our lead compound, ISP358-2, is selective for ERβ, and shows no obvious signs of peripheral toxicity. Importantly, ISP358-2 also enhances multiple types of memory dependent on the hippocampus, a brain region involved in numerous disorders including AD, depression, and schizophrenia.^{43, 46} ISP358-2 is distinct from previously reported ERβ agonists in that it has higher selectivity for ERβ over ERα, and in that it more closely resembles that naturally occurring 17β-estradiol molecule (FIG. 1d), as an A-C estrogen. Our studies also demonstrated biological efficacy in behavioral assays that were performed via three routes of administration: direct dorsal hippocampal infusion, intraperitoneal injection, and oral gavage, the latter two of which illustrate brain penetrance of the effective doses (FIG. 8). Overall, these findings suggest that the novel ERβ agonist ISP358-2 could be a promising drug candidate for enhancing memory in a variety of disorders characterized by memory dysfunction that occurs under low estrogen conditions, such as menopause.

Experimental Section

Compound Synthesis. All the chemicals were purchased from Sigma-Aldrich, Matrix Scientific, or Alfa Aesar and used as received. Reactions with moisture- or air-sensitive reagents were conducted under an inert atmosphere of nitrogen in oven-dried glassware with anhydrous solvents. Reactions were followed by TLC on precoated silica plates (60 Å, F₂₅₄, EMD Chemicals Inc) and were visualized by UV lamp (UVGL-25, 254/365 nm). Flash column chromatography was performed by using flash silica gel (32-63μ). NMR spectra were recorded on Varian UnityInova 400 MHz instrument. CDCl₃, d₆-acetone, and CD₃OD were purchased from Cambridge Isotope Laboratories. ¹H NMR spectra were calibrated to δ=7.26 ppm for residual CHCl₃, δ=2.05 ppm for d₅-acetone and δ=3.30 ppm for residual d₃-CD₃OD. ¹³C NMR spectra were calibrated from the central peak at δ=77.23 ppm for CDCl₃, δ=29.92 ppm for d₆-acetone and δ=49.00 ppm for CD₃OD. Purity of all compounds was >95%, determined with chromatography and NMR.

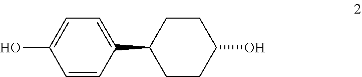 [SMILES: Oc1ccc([C@H]2CC[C@H](O)CC2)cc1]

trans-4-(4-Hydroxycyclohexyl)phenol (2). To a solution of 1 (0.200 g, 5.30 mmol) in anhydrous methanol (15 mL) at room temperature was added solid NaBH₄ (0.400 g, 10.6 mmol). The mixture was stirred for 3 h and then extracted with several times with ethyl acetate. The combined extracts were concentrated to give 2 (0.181 g, 90%) as a colorless solid. mp 196-208° C. ¹H NMR (400 MHz, CD₃OD) δ 7.00 and 6.67 (AA′ XX′, J_{AX}=8.7 Hz, 4H), 3.61-3.53 (m, 1H), 2.38 (tt, J=11.8, 3.4 Hz, 1H), 2.05-1.98 (m, 2H), 1.87-1.78 (m, 2H), 1.56-1.30 (m, 4H) ppm. ¹³C NMR (100 MHz, CD₃OD) 156.6, 139.2, 128.7, 116.1, 71.4, 49.3, 44.3, 36.9, 34.2 ppm. HRMS m/z 191.1077 [calcd for C₁₂H₁₅O₂⁻ (M-H⁺) 191.1077].

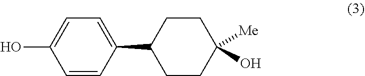

4-(4-Hydroxy-4-methylcyclohexyl)phenol (3). To a solution of 1 (0.100 g, 0.526 mmol) in dry ether (20 mL) at −78° C. under N₂, was slowly added a solution of methyllithium-lithium bromide complex (1.5 M in ether, 0.78 mL, 1.2 mmol). The mixture was stirred at −78° C. for 30 min, warmed to room temperature and stirred for another 1 h. The mixture was cooled to 0° C. and quenched with water. The mixture was extracted several times with ether, and the combined extracts dried (Na₂SO₄) and concentrated. The residue was purified by column chromatography (SiO₂, hexanes-ethyl acetate=4:1) to give 3 (0.040 g, 37%) as a colorless solid. mp 126-131° C.; ¹H NMR (400 MHz, CD₃OD) δ 7.03 and 6.67 (AA′XX′, J_{AX}=8.3 Hz, 4H), 2.35 (tt, J=12.4, 3.6 Hz, 1H), 1.87-1.69 (m, 4H), 1.61-1.44 (m, 4H) 1.21 (s, 3H); ¹³C NMR (100 MHz, CD₃OD) δ 156.5, 140.0, 128.8, 116.1, 69.5, 44.5, 40.0, 31.9, 31.1 ppm. HRMS m/z 205.1234 [calcd for C₁₃H₁₇O₂⁻ (M-H⁺) 205.1234]. Anal. calcd. for C₁₃H₁₈O₂: C, 75.69; H 8.79. Found: C, 75.33; H, 8.83.

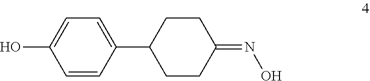 [SMILES: ON=C1CCC(c2ccc(O)cc2)CC1]

4-(4-Hydroxyphenyl)cyclohexanone oxime (4). To a solution of 1 (0.050 g, 0.26 mmol) in ethanol (10 mL), were added Amberlyst (0.060 g) and hydroxylamine hydrochloride (0.039 g, 0.560 mmol). The mixture was stirred at room temperature for 2 h and then filtered. The filtrate was concentrated and extracted several times with ethyl acetate. The combined organic extracts were washed with water, dried (MgSO₄), and concentrated to give 4 as a colorless solid (0.037 g, 70%). mp 171-174° C.; ¹H NMR (400 MHz, CD₃OD) δ 7.00 and 6.69 (AA′XX′, J_{AX}=8.2 Hz, 4H), 3.39 (broad d, J=13.5 Hz, 1H), 2.67 (t, J=12.8 Hz, 1H), 2.41 (broad d, J=14.0 Hz, 1H), 2.20 (td, J=14.6, 5.4 Hz, 1H), 1.93 (broad t, J=15.8 Hz, 2H), 1.81 (td, J=14.0, 5.2 Hz, 1H), 1.61-1.42 (m, 2H) ppm. ¹³C NMR (100 MHz, CD₃OD) δ 160.8, 156.7, 138.3, 128.6, 116.2, 44.1, 35.8, 34.6, 32.8, 25.1 ppm. Anal. calcd. for C₁₂H₁₅NO₂: C, 70.22; H 7.36; N, 6.83. Found: C, 69.93; H, 7.36; N, 6.63.

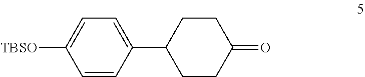 [SMILES: CC(C)(C)[Si](C)(C)Oc1ccc(C2CCC(=O)CC2)cc1]

4-(4-t-Butyldimethylsilyloxyphenyl)cyclohexan-1-one (5). To a solution of 1 (0.500 g, 2.62 mmol) in anhydrous CH₂Cl₂ (30 mL) at 0° C. under N₂, was added imidazole (0.357 g, 5.24 mmol). After 30 min t-butyldimethylsilyl chloride (0.594 g, 3.94 mmol) was added and the mixture was gradually warmed to room temperature overnight. The resulting mixture was diluted with brine (25 mL) and extracted several times with CH₂Cl₂. The combined organic extracts were dried (Na₂SO₄) and concentrated. The residue was purified by column chromatography (SiO₂, hexanes-ethyl acetate=9:1) to give 5 (0.664, 83%) as a colorless solid. mp 39-42° C. ¹H NMR (400 MHz, CDCl₃) δ 7.08 and 6.78 (AA′XX′, J_{AX}=8.4 Hz, 4H), 2.96 (t, J=12.3 Hz, 1H), 2.56-2.40 (m, 4H), 2.25-2.14, (m, 2H), 1.97-1.82 (m, 2H), 0.98 (s, 9H), 0.19 (s, 6H) ppm. ¹³C NMR (100 MHz, CDCl₃) δ 211.6, 154.3, 137.7, 127.7, 120.1, 42.2, 41.6, 34.6, 25.9, 18.4, −4.2 ppm.

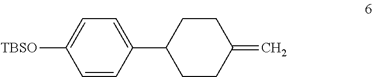 [SMILES: C=C1CCC(c2ccc(O[Si](C)(C)C(C)(C)C)cc2)CC1]

t-Butyldimethyl(4-(4-methylenecyclohexyl)phenoxy)silane (6). To a solution of methyltriphenylphosphonium bromide (0.836 g, 2.34 mmol) in dry THF (20 mL) at −10° C. under N₂, was slowly added a solution of n-butyllithium (1.6 M in hexane, 1.50 mL, 2.4 mmol). After 30 min, a solution of 5 (0.502 g, 1.17 mmol) in dry THF (8 mL) was added dropwise. The reaction mixture was slowly warmed to room temperature and stirred overnight. After this time, the mixture was diluted with water (20 mL), extracted several times with ethyl acetate, and the combined extracts were dried (Na₂SO₄) and concentrated. Purification of the crude residue by column chromatography (SiO₂, hexanes-ethyl acetate=9:1) gave 6 (1.678 g, 84%) as a colorless oil. ¹H NMR (400 MHz, CDCl₃) δ 7.06 and 6.77 (AA′XX′, J_{AX}=8.3 Hz, 4H), 4.68 (s, 2H), 2.62 (tt, J=12.1, 3.4 Hz, 1H), 2.42 (broad d, J=13.5 Hz, 2H), 2.18 (broad t, J=13.2, 2H), 2.00-1.93 (m, 2H), 1.57-1.45 (m, 2H), 0.99 (s, 9H), 0.20 (s, 6H) ppm. ¹³C NMR (100 MHz, CDCl₃) δ 153.9, 149.2, 139.8, 127.8, 119.9, 107.4, 43.5, 35.9, 35.4, 25.9, 18.4, −4.2 ppm. Anal. calcd. for C₁₉H₃₀O₂Si: C, 75.43; H, 9.99. Found: C, 75.71; H, 10.02.

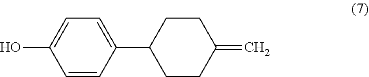

4-(4-Hydroxyphenyl)methylenecyclohexane (7). To a solution of 6 (0.739 g, 0.244 mmol) in anhydrous THF (20 mL) was added a solution of TBAF (1 M in THF, 9.8 mL, 9.8 mmol). The mixture was heated at reflux for 5 h. After cooling, the solution was partitioned between ethyl acetate and water, and the aqueous layer was extracted several times with ethyl acetate. The combined organic layers were washed with brine, dried (Na₂SO₄) and concentrated. Purification of the residue by column chromatography (SiO₂, hexanes-ethyl acetate=4:1) gave 7 (0.379 g, 83%) as a colorless solid. mp 82-84° C.; ¹H NMR (400 MHz, CD₃OD) δ 6.99 and 6.67 (AA′XX′, J_{AB}=8.5 Hz, 4H), 4.63 (t, J=1.7 Hz, 2H), 2.57 (tt, J=12.3, 4.3 Hz, 1H), 2.41-2.33 (m, 2H), 2.22-2.11 (m, 2H), 1.94-1.85 (m, 2H), 1.45 (qd, J=12.3, 4.3 Hz, 2H); ¹³C NMR (100 MHz, CD₃OD) δ 156.6, 150.3, 139.3, 128.8, 116.2, 107.8, 44.8, 37.3, 36.4 ppm. HRMS m/z 187.1128 [calcd for C₁₃H₁₅O⁻ (M-H⁺) 187.1128]. Anal. calcd. for C₁₃H₁₆O₂: C, 82.93; H 8.57. Found: C, 82.71; H, 8.58.

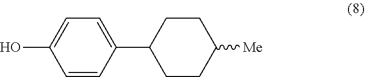 [SMILES: CC1CCC(c2ccc(O)cc2)CC1]

4-(4-Methylcyclohexyl)phenol (8). To a solution of 7 (0.150 g, 0.797 mmol) in methanol (10 mL) was added 10% Pd/C (85 mg, 10 mol %). The mixtures was stirred under a balloon filled with H₂, at room temperature, for 12 h. The reaction mixture was filtered through a pad of celite, dried (Na₂SO₄) and concentrated. The residue was purified by column chromatography (SiO₂, hexanes-ethyl acetate=4:1) to give 8 (0.121 g, 80%) as a colorless solid. This was determined to be a mixture of cis- and trans-stereoisomers by ¹H NMR spectroscopy. mp 93-99° C.; ¹H NMR (400 MHz, CD₃OD) 7.05-6.96 (m, 2H), 6.70-6.64 (m, 2H), 2.48-2.28 (m, 1H), 1.83-1.34 (m, 8H), 1.13-1.04 (m, 1H), 1.03 (d, J=7.2 Hz, 1H), 0.92 (d, J=6.6 Hz, 2H); ¹³C NMR (100 MHz, CD₃OD) δ 156.3, 140.0, 128.6, 116.0, 44.7, 36.9, 35.9, 33.7, 33.1, 30.1, 23.1 ppm.

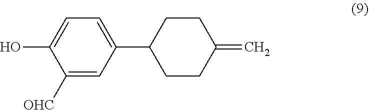 [SMILES: C=C1CCC(c2ccc(O)c(C=O)c2)CC1]

2-Hydroxy-5-(4-methylenecyclohexyl)benzaldehyde (9). To a solution of 7 (0.100 g, 0.531 mmol) in dry CH₃CN (20 mL) were sequentially added MgCl₂ (0.076 g, 0.797), triethylamine (0.28 mL, 2.0 mmol), followed by paraformaldehyde (0.108 g, 3.59 mmol). The mixture was heated at reflux for 6 h. The mixture was cooled to room temperature and quenched with 10% HCl (10 mL) and extracted several times with ethyl acetate. The combined extracts were washed with brine, dried (Na₂SO₄) and concentrated. Purification of the residue by column chromatography (SiO₂, hexanes-diethyl ether=4:1) gave 9 (0.046 g, 40%) as a colorless oil. ¹H NMR (400 MHz, CD₃OD) δ 9.96 (s, 1H), 7.50 (s, 1H), 7.39 (d, J=8.5 Hz, 1H), 6.85 (d, J=8.5 Hz, 1H), 4.65 (t, J=1.7 Hz, 2H), 2.68 (tt, J=12.0, 3.4 Hz, 1H), 2.44-2.34 (m, 2H), 2.24-2.13 (m, 2H), 1.97-1.89 (m, 2H), 1.49 (qd, J=13.0, 4.0 Hz, 2H); ¹³C NMR (100 MHz, CD₃OD) δ 197.5, 161.0, 149.8, 139.9, 137.0, 131.6, 122.5, 118.2, 108.2, 44.3, 36.9, 36.2 ppm. HRMS m/z 231.1027 [calcd for C₁₄H₁₅O₃ (M-H⁺) 231.1027].

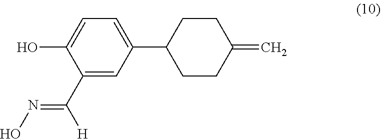 [SMILES: C=C1CCC(c2ccc(O)c(/C=N/O)c2)CC1]

2-Hydroxy-5-(4-methylenecyclohexyl)benzaldehyde oxime (10). To a solution of 9 (0.050 g, 0.232 mmol) in pure ethanol (10 mL), were added sodium bicarbonate (0.024 g, 0.278 mmol) and hydroxylamine hydrochloride (0.025 g, 0.348 mmol). The reaction was heated at 80° C. for 5 h and the mixture was extracted several times with ethyl acetate. The combined organic extracts were dried (MgSO₄) and concentrated. Purification of the residue by column chromatography (SiO₂, hexanes-ethyl acetate=13:7) gave 10 (0.037 g, 69%) as a colorless solid. mp 120-125° C.; ¹H NMR (400 MHz, CD₃OD) δ 8.20 (s, 1H), 7.09-7.06 (m, 1H), 7.05 (d, J=2.4 Hz, 1.8H), 6.99 (d, J=7.9 Hz, 0.2H), 6.78 (d, J=8.1 Hz, 0.8H), 6.68 (d, J=8.6 Hz, 0.2H), 4.63 (t, J=1.6 Hz, 2H), 2.60 (tt, J=12.2, 3.3 Hz, 1H), 2.42-2.33 (m, 2H), 2.22-2.10 (m, 2H), 1.94-1.85 (m, 2H), 1.46 (qd, J=12.5, 4.0 Hz, 2H); ¹³C NMR (100 MHz, CD₃OD) δ 156.6, 152.4, 150.1, 139.4, 130.2, 129.2, 128.7, 118.5, 117.2, 116.2, 108.0, 107.7, 44.5, 37.3, 37.1, 36.4, 36.3 ppm. HRMS m/z 230.1187 [calcd for C₁₄H₁₆NO₃ (M-H⁺) 230.1186].

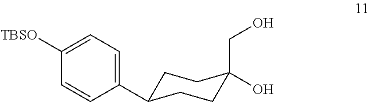 [SMILES: CC(C)(C)[Si](C)(C)Oc1ccc(C2CCC(O)(CO)CC2)cc1]

4-(4-((t-Butyldimethylsilyl)oxy)phenyl)-1-(hydroxymethyl)cyclohexan-1-01 (11). To a solution of 6 (0.280 g, 0.926 mmol) and N-methylmorpholine-N-oxide (0.13 mL, 1.3 mmol) in acetone (6 mL) and distilled water (0.3 mL) was added a solution of OsO₄ in tert-butanol (2.5%, 90 μL). The mixture was stirred overnight and saturated aqueous NaHSO₃ (10 mL) was added to quench the reaction. The mixture was diluted with ether and washed several times with water. The organic layer was dried (MgSO₄), concentrated and the residue purified by column chromatography (SiO₂, hexanes-ethyl acetate=1:4) to give 11 (0.267 g, 86%) as a colorless solid. mp 80-86° C.; ¹H NMR (400 MHz, CDCl₃) δ 7.04 and 6.75 (AA′XX′, J_{AX}=8.5 Hz, 4H), 3.69 (s, 2H), 2.52 (tt, J=11.4, 3.6 Hz, 1H), 2.04-1.72 (m, 4H, solvent peak overlap), 1.61-1.37 (m, 4H), 0.97 (s, 9H), 0.18 (s, 6H); ¹³C NMR (100 MHz, CDCl₃) δ 154.0, 138.9, 127.7, 120.0, 72.4, 66.2, 42.8, 35.4, 31.3, 25.9, 18.4, −4.2 ppm.

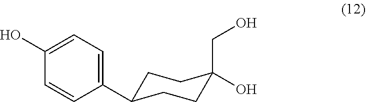 [SMILES: OCC1(O)CCC(c2ccc(O)cc2)CC1]

4-(4-Hydroxy-4-(hydroxymethyl)cyclohexyl)phenol (12). To a solution of 11 (0.230 g, 0.683 mmol) in anhydrous THF (10 mL) was added a solution of TBAF (1M in THF, 2.8 mL, 2.8 mmol). The mixture was heated at reflux for 6 h and cooled to room temperature. The solution was partitioned between ethyl acetate and water. The combined organic layers were washed with brine, dried (Na₂SO₄) and concentrated. Purification of the residue by column chromatography (SiO₂, ethyl acetate-methanol=9:1) gave 12 (0.118 g, 78%) as a colorless solid. mp 182-188° C.; ¹H NMR (400 MHz, CD₃OD) δ 7.02 and 6.68 (AA′XX′, J_{AX}=8.5 Hz, 4H), 3.62 (s, 2H), 2.53-2.42 (m, 1H), 1.99-1.89 (m, 2H), 1.85-1.68 (m, 2H), 1.58-1.43 (m, 4H); ¹³C NMR (100 MHz, CD₃OD) δ 156.6, 138.9, 128.8, 116.2, 73.1, 66.6, 44.3, 36.0, 32.6 ppm. Anal. calcd. for C₁₃H₁₈O₃: C, 70.24; H, 8.16. Found: C, 70.18; H, 7.78.

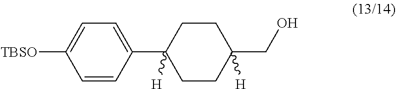 [SMILES: CC(C)(C)[Si](C)(C)Oc1ccc(C2CCC(CO)CC2)cc1]

4-(4-t-Butyldimethylsilyloxyphenyl)cyclohexyl)methanol (13/14). To a solution of 6 (0.821 g, 2.71 mmol) in THF (24 mL) at 0° C. under N₂, was added a solution of borane-THF complex (1 M in THF, 5.4 mL, 5.4 mmol). The reaction mixture was slowly warmed to room temperature and stirred for 20 h. The mixture was then cooled to 0° C., followed by sequential addition of ethanol (50 mL), hydrogen peroxide solution (30% in water, 4.0 mL) and 1N NaOH solution (20 mL). The mixture was warmed to room temperature and stirred for 90 min. The reaction mixture was quenched with saturated sodium bicarbonate solution (10 mL), diluted with water (20 mL) and extracted with several times with ethyl acetate. The combined organic extracts were washed with brine, dried (Na₂SO₄,) and concentrated. Purification of the residue by column chromatography (SiO₂, hexanes-ethyl acetate=7:3) gave a colorless oil (0.572 g, 66%). This was determined to be a 2:1 mixture of cis-13 and trans-14 by ¹H NMR integration of the signals for the CH₂OH groups at δ 3.69 and 3.50 ppm respectively. ¹H NMR (400 MHz, CDCl₃) δ 7.06 and 6.76 (AA′XX′, J_{AX}=8.5 Hz, 4H), 3.69 (d, J=7.4 Hz, 1.3H), 3.50 (d, J=6.5 Hz, 0.7H), 2.59-2.51 (m, 0.5H), 2.42 (tt, J=12.1, 3.8 Hz, 0.5H), 1.96-1.84 (m, 2H), 1.80-1.37 (m, 7H), 0.98 (s, 9H), 0.19 (s, 6H) ppm. ¹³C NMR (100 MHz, CDCl₃) δ 153.7, 140.4, 140.0, 127.8, 119.9, 68.9, 64.6, 43.8, 42.6, 40.3, 36.2, 34.1, 30.0, 29.4, 27.0, 25.9, 18.4, −4.2 ppm. Use of 9-BBN instead of BH₃-THF gave a 2:3 mixture of cis-13:trans-14 (74%).

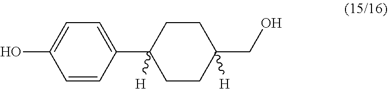 [SMILES: OCC1CCC(c2ccc(O)cc2)CC1]

4-(4-(Hydroxymethyl)cyclohexyl)phenol (15/16). To a solution of 13/14 (0.594 g, 1.85 mmol, 2:1 mixture c:t) in dry THF (10 mL) was added a solution of TBAF (1 M in THF, 7.5 mL, 7.5 mmol). The reaction mixture was heated to reflux at 70° C. overnight and cooled to room temperature. The solution was partitioned between ethyl acetate and water. The organic layer was washed with brine, dried (Na₂SO₄) and concentrated. The residue was purified by column chromatography (SiO₂, hexanes-ethyl acetate=3:2) to give a colorless solid (0.280 g, 73%). This was determined to be a 2:1 mixture of cis-13 and trans-14 stereoisomers by ¹H NMR integration of the signals for the CH₂OH groups at δ 3.60 and 3.39 ppm respectively. mp 118-122° C. ¹H NMR (400 MHz, CD₃OD) δ 7.04-6.98 (m, 2H), 6.70-6.65 (m, 2H), 3.60 (d, J=7.6 Hz, 1.5H), 3.39 (d, J=6.6 Hz, 0.5H), 2.54-2.44 (m, 1H), 2.37 (tt, J=12.1, 3.4 Hz, 1H), 1.93-1.70 (m, 3H), 1.61 (d, J=6.3 Hz, 4H), 1.46-1.37 (m, 1H), 1.14-1.02 (m, 1H) ppm. ¹³C NMR (100 MHz, CD₃OD) δ 156.2, 139.6, 128.7, 116.0, 68.0, 64.4, 45.2, 44.0, 41.4, 37.0, 35.4, 31.2, 30.5, 28.0 ppm.

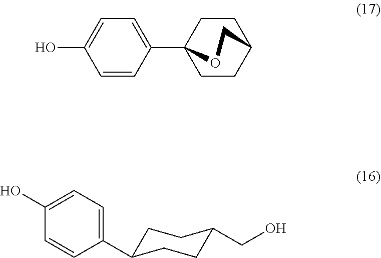

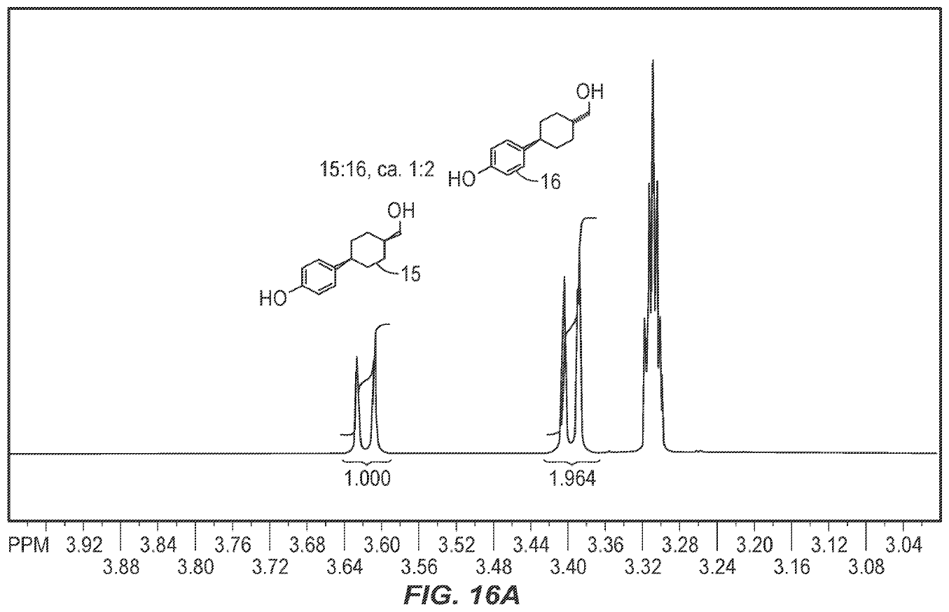

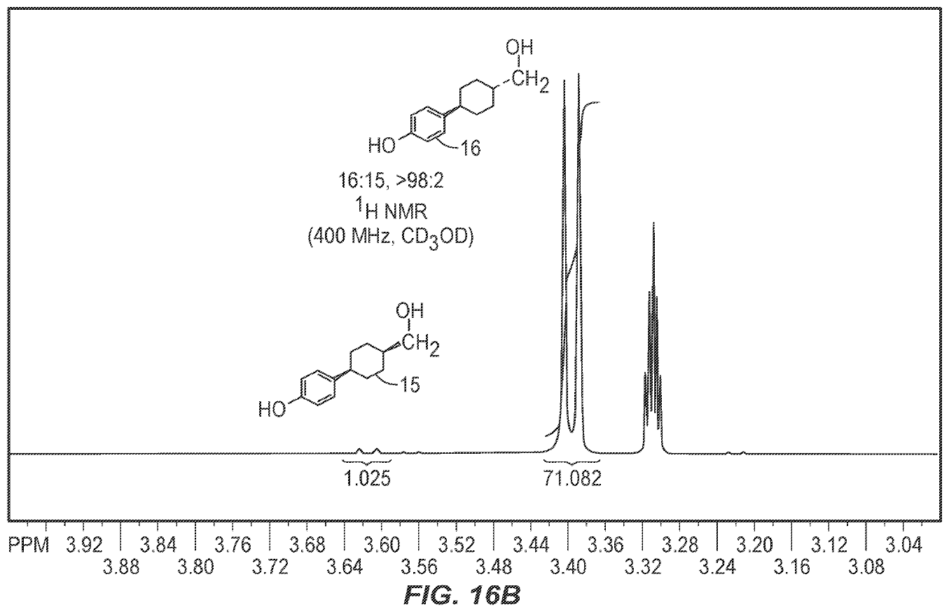

1-(4-Hydroxyphenyl)-2-oxabicyclo[2.2.2]octane (17) and trans-(hydroxymethyl)cyclohexyl) phenol (16). To a solution of 15/16 (0.080 g, 0.388 mmol, 2:3 mixture of cis-15:trans-16) in anhydrous CH₂Cl₂ (20 mL) at −10° C., was slowly added, over a period of 30 min, a suspension of 2,3-dichloro-5,6-dicyano-1,4-benzoquinone (0.044 g, 0.194 mmol) in CH₂Cl₂ (4 mL). The green solution was stirred at 0° C. for 2 h and gradually warm to room temperature and stirred for another 3 h. The mixture was quenched by slow addition of saturated sodium bicarbonate solution at 0° C. After a 10 min the layers were separated and aqueous layer was extracted several times with CH₂Cl₂. The combined organic extracts were washed with brine, dried (Na₂SO₄), and concentrated. The residue was purified by column chromatography (SiO₂, hexanes-ethyl acetate=3:2) to give 17 (0.029 g, 37%) followed by 16 (0.038 g, 47%) both as colorless solids. Purity of 16 was established by ¹H NMR (FIG. 16).

17: mp 120-124° C.; ¹H NMR (400 MHz, CD₃OD) δ 7.18 and 6.64 (AA′XX′, J_{AX}=7.9 Hz, 4H), 4.04 (s, 2H), 2.01 (t, J=7.8 Hz, 4H), 1.94-1.73 (m, 5H); ¹³C NMR (100 MHz, CD₃OD) δ 157.3, 139.0, 127.2, 115.8, 73.2, 71.5, 34.7, 27.5, 26.1 ppm. Anal. calcd. for C₁₃H₁₆O₂: C, 76.44; H, 7.89. Found: C, 76.39; H, 7.97.

16: mp 115-120° C.; ¹H NMR (400 MHz, CD₃OD) δ 7.00 and 6.68 (AA′XX′, J_{AX}=8.7 Hz, 4H), 3.39 (d, J=6.7 Hz, 2H), 2.36 (tt, J=12.1, 3.0 Hz, 1H), 1.87 (broad t, J=15.4, 4H), 1.55-1.36 (m, 3H), 1.14-1.02 (m, 2H); ¹³C NMR (100 MHz, CD₃OD) δ 156.5, 140.0, 128.7, 116.1, 68.9, 45.3, 41.5, 35.5, 31.3 ppm. Anal. calcd. for C₁₃H₁₈O₂: C, 75.69; H, 8.79. Found: C, 75.66; H, 9.09.

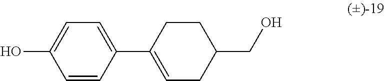 [SMILES: OCC1CC=C(c2ccc(O)cc2)CC1]

4′-(Hydroxymethyl)-2′,3′,4′,5′-tetrahydro-[1,1′-biphenyl]-4-ol (±)-19. To a solution of 17 (0.103 g, 0.504 mmol) in dry CH₃CN (25 mL) was added MgCl₂ (0.072 g, 0.756 mmol) followed by triethylamine (0.26 mL, 1.89 mmol). The mixture was heated at reflux for 8 h, then cooled and quenched with 10% HCl (15 mL). The mixture was extracted several times with ethyl acetate, and the combined extracts washed with brine, dried (Na₂SO₄) and concentrated. Purification of the residue by column chromatography (SiO₂, hexanes-ethyl acetate=13:7) gave 19 (0.080 g, 78%) as a colorless solid. mp 177-184° C.; ¹H NMR (400 MHz, CD₃OD) δ 7.20 and 6.69 (AA′XX′, J_{AX}=8.6 Hz, 4H), 5.97-5.92 (m, 1H), 3.48 (dd, J=6.4, 2.6 Hz, 2H), 2.49-2.23 (m, 3H), 2.01-1.71 (m, 4H), 1.43-1.31 (m, 1H); ¹³C NMR (100 MHz, CD₃OD) δ 157.5, 137.7, 135.3, 127.2, 122.1, 116.0, 68.0, 37.5, 30.1, 28.2, 27.2 ppm. HRMS m/z 203.1078 [calcd for C₁₃H₁₅O₂⁻ (M-H⁺) 203.1077].

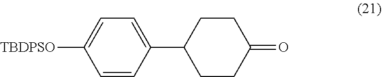

4-(4-((t-Butyldiphenylsilyl)oxy)phenyl)cyclohexan-1-one (21). To a solution of 1 (0.815 g, 4.28 mmol) in dry CH₂Cl₂ (30 mL) at 0° C., was added imidazole (0.583 g 8.57 mmol) followed by dropwise addition of a solution of t-butyldiphenylsilyl chloride (1.60 mL, 5.57 mmol) in CH₂Cl₂ (9 mL). The reaction mixture was slowly warmed to room temperature and stirred for 12 h. The mixture was diluted with water and extracted several times with CH₂Cl₂. The combined extracts were washed with brine, dried (Na₂SO₄), and concentrated. The residue was purified by column chromatography (SiO₂, hexanes-ethyl acetate=4:1) to give 21 (1.70 g, 93%) as a colorless solid. mp 83-84° C.; ¹H NMR (400 MHz, CDCl₃) δ 7.74-7.70 (m, 4H), 7.45-7.34 (m, 6H), 6.96 and 6.71 (AA′BB′, J_{AB}=8.6 Hz, 4H), 2.90 (tt, J=12.1, 3.3 Hz, 1H), 2.49-2.42 (m, 4H), 2.19-2.10 (m, 2H), 1.91-1.77 (m, 2H), 1.09 (s, 9H); ¹³C NMR (100 MHz, CDCl₃) δ 211.6, 154.3, 137.3, 135.7, 133.2, 130.1, 127.9, 127.5, 119.8, 42.1, 41.6, 34.3, 26.7, 19.7 ppm.

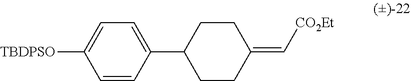

Methyl 2-(4-(4-t-butyldiphenylsilyloxyphenyl)cyclohexylidene)acetate (±)-22. To a solution of trimethyl phosphonoacetate (0.160 mL, 0.980 mmol) in dry THF (5 mL) at 0° C., was added NaH (40 mg, 55% in mineral oil, 0.980 mmol). After stirring for 45 min, a solution of 21 (0.350 g, 0.816 mmol) in dry THF (5 mL) was added and the mixture was warmed to room temperature and stirred for 8 h. The mixture was diluted with water and extracted several times with ether. The combined extracts were dried (MgSO₄) and concentrated. The residue was purified by column chromatography (SiO₂, hexanes-ethyl acetate=9:1) to give 22 (0.376 g, 95%) as colorless gum. ¹H NMR (400 MHz, CDCl₃) δ 7.74-7.68 (m, 4H), 7.44-7.32 (m, 6H), 6.91 and 6.69 (AA′XX′, J_{AX}=8.6 Hz, 4H), 5.65 (s, 1H), 3.96-3.88 (m, 1H), 3.69 (s, 3H), 2.66 (tt, J=12.1, 3.4 Hz, 1H), 2.38-2.24 (m, 2H), 2.04-1.93 (m, 3H), 1.59-1.46 (m, 2H), 1.08 (s, 9H); ¹³C NMR (100 MHz, CDCl₃) δ 167.4, 162.7, 154.0, 138.6, 135.7, 133.3, 130.0, 127.9, 127.5, 119.6, 113.3, 51.10, 43.3, 37.9, 35.9, 35.1, 29.7, 26.7, 19.7 ppm.

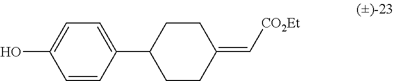 [SMILES: CCOC(=O)C=C1CCC(c2ccc(O)cc2)CC1]

4-[(4-Hydroxyphenyl)cyclohexylidene]acetic acid ethyl ester (±)-23. To a stirring solution of 22 (60 mg, 0.12 mmol) in dry THF (1 mL) was added a solution of tetrabutylammonium fluoride (0.247 mL, 1.0 M in THF, 0.247 mmol). The solution was stirred at room temperature after 1 h, and then the mixture was diluted with water and extracted with ethyl acetate. The combined extracts were washed with brine, dried and concentrated. The residue was purified by preparative TLC (SiO₂, hexanes-ethyl acetate=9:1) to give 23 (20 mg, 64%) as a colorless solid. mp 92-94° C.; ¹H NMR (CDCl₃, 300 MHz) δ 7.08 and 6.77 (AA′XX′, J_{AB}=8.4 Hz, 4H), 5.68 (s, 1H), 4.58 (s, 1H), 4.17 (q, J=7.1 Hz, 2H), 4.00-3.90 (m, 1H), 2.80-2.68 (m, 1H), 2.45-1.97 (m, 6H), 1.30 (t, J=7.3 Hz, 3H); ¹³C NMR (CDCl₃, 75 MHz) δ 167.0, 162.2, 154.0, 138.6, 128.1, 115.4, 113.9, 59.8, 43.4, 37.9, 36.0, 35.2, 29.7, 14.5 ppm. HRMS m/z 259.1339 [calcd for C₁₆H₁₉O₃⁻ (M-H⁺) 259.1340].

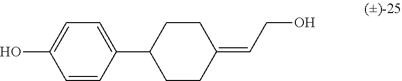 [SMILES: OCC=C1CCC(c2ccc(O)cc2)CC1]

4-(4′-Hydroxyphenyl)(2-hydroxyethylidene)cyclohexane (±)-25. To a solution of 22 (275 mg, 0.551 mmol) in dry CH₂Cl₂ (2 mL) under N₂ at −40° C. was added a solution of diisobutylaluminum hydride (1.0 M in CH₂Cl₂, 1.41 mL, 1.41 mmol). After 90 min, saturated aqueous potassium sodium tartrate was added and reaction mixture warmed to room temperature. After 2 h the layers were separated and the aqueous layer was extracted several times with CH₂Cl₂. The combined organic layers were dried, filtered through a pad of celite and concentrated to give 4-(4′-t-butyldiphenylsilyloxyphenyl)(2-hydroxyethylidene)cyclohexane (254 mg, quantitative) as a colorless gum. This compound was used without further purification. To a solution of the crude allylic alcohol (235 mg, 0.514 mmol) in dry THF (1 mL) under nitrogen was added a solution of tetrabutylammonium fluoride in (1.0 M in THF, 1.03 mL, 1.03 mmol). The solution was stirred for 3 h and then diluted with water and the resultant mixture extracted several times with ethyl acetate. The combined extracts were washed with brine, dried and concentrated. The residue was purified by column chromatography (SiO₂, hexanes-ethyl acetate=4:1) to give 25 (90 mg, 80%) as a colorless solid. mp 165-166° C.; ¹H NMR (d₆-acetone, 300 MHz) δ 8.10 (s, 1H), 7.04 and 6.74 (AA′XX′, J_{AX}=8.4 Hz, 4H), 5.36 (t, J=6.6 Hz, 1H), 4.17-4.02 (m, 2H), 2.78-2.70 (m, 1H), 2.64 (tt, J=3.3, 12.0 Hz, 1H), 2.35-2.10 (m, 2H), 1.98-1.80 (m, 4H), 1.54-1.37 (m, 2H); ¹³C NMR (d₆-acetone, 75 MHz) δ 156.5, 141.1, 138.6, 128.5, 123.6, 116.0, 58.5, 44.6, 37.5, 37.0, 36.2, 29.2. Anal. calcd. for C₁₄H₁₈O₂: C, 77.03; H 8.31. Found: C, 77.20; H, 8.28.

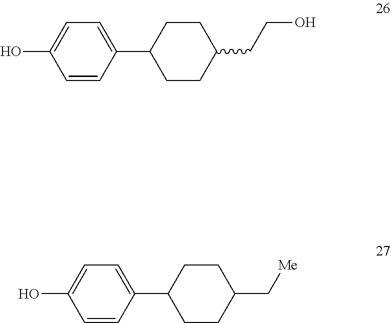 [SMILES: OCCC1CCC(c2ccc(O)cc2)CC1 ; CCC1CCC(c2ccc(O)cc2)CC1]

4-[4-(2-Hydroxyethyl)cyclohexyl]phenol (26) and 4-(4-ethylcyclohexyl)phenol (27). A solution of 25 (50 mg, 0.23 mmol) in methanol (15 mL) with small pinch of 20% Pd/C was stirred under H₂ (30 psi) for 12 h. The reaction mixture was filtered through a pad of celite, concentrated and the residue was purified by preparative TLC (SiO₂, hexanes-ethyl acetate=13:7) to give 27 (28 mg, 60%), followed by 26 (7 mg, 14%) both as colorless solids.

27: mp 120-125° C.; ¹H NMR (d₆-acetone, 300 MHz) δ 8.02 (s, 1H), 7.08-7.01 (m, 2H), 6.77-6.71 (m, 2H), 3.65-3.56 and 3.43-3.37 (m, 3H total), 2.52-2.33 (m, 1H), 1.91-1.00 (m, 11H); ¹³C NMR (d₆-acetone, 75 MHz) δ 156.4, 139.5, 128.6, 115.9, 61.1, 60.4, 44.6, 41.4, 35.5, 34.8, 34.5, 31.1, 30.3 ppm. HRMS m/z 219.139 [calcd for O₁₄H₁₉O₂⁻ (M-H⁺) 219.1390].

26: mp 80-81° C.; ¹H NMR (CDCl₃, 300 MHz) δ 7.08 and 6.76 (AA′XX′, J_{AX}=8.1 Hz, 4H), 4.55 (s, 1H), 2.54-2.35 (m, 1H), 1.92-1.82 (m, 2H), 1.70-1.50 (m, 3H), 1.45-1.00 (m, 6H), 0.91 (t, J=7.2 Hz, 3H) ppm.

TR-FRET Assay. LanthaScreen® TR-FRET ER Alpha and Beta Competitive Binding Assay kits from Thermo Fisher Scientific were used to perform the TR-FRET assays. These included a terbium-labeled anti-GST antibody, a fluorescent small molecule ER Alpha or Beta ligand as a “tracer”, and a human ER Alpha or Beta ligand-binding domain (LBD) that is tagged with glutathione-S-transferase (GST) in a homogenous mix-and-read assay format.

The TR-FRET assay employs a Tb-anti-GST antibody that binds to a GST tag, and a fluorescently labeled estrogen (tracer) binds in the active site pocket. The TR-FRET signal obtained decreases when competitor compounds displace the fluorescently labeled tracer. Assays were performed according to kit instructions. Briefly, 1:5 dilution series of compounds were made with DMSO, then diluted in assay buffer such that the highest concentration tested in the assay was 50 μM for ERβ and 50 μM for ERα and DMSO was 1%. Assays were set up in 384-well white, small volume plates (Corning® 4512). The assay was incubation for 1 hour in the dark at room temperature, after which plates were spun at 1000 rpm in a tabletop centrifuge equipped with a swing-out rotor (Eppendorf 5810, rotor A-4-64). TR-FRET signal was read on a SpectraMax M5 (Molecular Devices) set-up according to Thermo Fisher Scientific machine settings (excitation of 332 nm, emissions 518 nm and 488 nm with a 420 nm cutoff, 50 μs integration delay, 400 μs integration time, and 100 flashes per read). The TR-FRET ratio was calculated using the SoftmaxPro software by dividing the emission at 518 nm (fluorescein) by the emission at 488 nm (Terbium). Data were normalized to E₂, which had an IC₅₀ of 0.25±0.06 nM in this assay. Data analysis was done using Prism (GraphPad Software, Inc., La Jolla, Calif.), with fits typically constrained to go to zero at high concentrations of competing ligand. Standard deviations are for the nonlinear least squares fit of the data. When replicate assays and fits were done, curves are shown (FIG. 2 and FIG. 9); and, fitted IC₅₀'s summarized in Table 1) for curves that gave median IC₅₀ values.

Nuclear Hormone Receptor Specificity Assay. Selectivity measurements were performed using the SelectScreen™ cell-based nuclear receptor profiling service from ThermoFisher (FIG. 3). This is a FRET-based assay that uses GeneBLAzer™ technology. It detects ligand binding to and activation of a nuclear hormone receptor of interest (ligand binding domain; LBD) that is fused to a GAL4 DNA binding domain (DBD), which upon activation induces expression of beta-lactamase. The assay has a Z′>0.5 in agonist mode. Compound stocks were in DMSO, and diluted for assay concentrations of 0.25, 2.5 and 25 μM. Data for estrogen receptors were normalized to E₂, which had an IC₅₀ of 0.107 nM for ERα and 0.579 nM for ERβ. Data for other receptors were normalized to an appropriate control, which are listed with IC₅₀ values in Table 2.

TABLE 2
Control compounds and IC₅₀ values for
the nuclear hormone specificity assay.

| | Compound | IC₅₀ |
|---|---|---|
| Nuclear Receptor | Name | (nM) |
| Androgen Receptor (AR) | R1881 | 0.302 |
| Glucocorticoid Receptor (GR) | Dexamethasone | 2.35 |
| Mineralocorticoid Receptor (MR) | Aldosterone | 0.305 |
| Peroxisome Proliferator-Activated | L-165041 | 12.6 |
| Receptor (PPARδ) | | |
| Progesterone Receptor (PR) | R5020 | 0.236 |
| Thyroid Hormone Receptor (TRβ) | T3 Free Acid | 0.103 |
| Vitamin D Receptor (VDR) | Calcitriol | 0.0953 |

Repeat assays for ERα and ERβ agonist assays in a 10-point curve were also completed (FIG. 3b). Data again were normalized to E₂, which had IC₅₀ values of 0.151 nM and 0.568 nM for ERα and ERβ, respectively.

Coactivator Assay. The LanthaScreen® TR-FRET assay from ThermoFisher was used (FIG. 4a), similar to the assay described above (FIG. 3); except, the LanthaScreen® assay has a fluorescently labeled coactivator peptide present. The assay measures recruitment of the labeled coactivator peptide to the ERα or En LBD, induced by the binding of the ER agonist being assayed. The coactivator peptide is PGC1a, derived from the PPARγ coactivator protein 1a, and containing an LXXLL motif (Sequence: EAEEPSLLKKLLLAPANTQ (SEQ ID NO:1). Data were normalized to E₂, which had an IC₅₀ of 2.58 nM and 2.79 nM for ERα and ERβ, respectively.

Cell-based Assays. ERα and ERβ cell-based assays for both agonist and antagonist activity measurements were performed using kits provided by Indigo Biosciences (FIG. 5). Assays relied on a luciferase reporter gene that was downstream from either an ERα or En-responsive promoter, and activated by an added agonist; or, had agonist activity blocked by an added antagonist. ER-induced luciferase expression was quantified using chemiluminescence, measured using a SpectraMax M5 plate reader. Stock solutions of ligands were prepared in DMSO and diluted to final concentrations (typically low nM to μM), using the Compound Screening Medium provided in the kit, such that the DMSO concentration in the assay was kept below the assay limit of 0.4%. Vehicle controls were included in both agonist and antagonist assays. Assays were conducted according to kit instructions. Briefly, cells directly from the freezer were diluted in Cell Recovery Media (provided) and warmed for 5 minutes at 37° C. The cell suspension was divided in half. Estradiol, E₂, was added to one half of the cells for antagonist assays while the remaining cells without E₂ were used for the agonist assay. Cells were plated and compounds to be screened were added. Plates were incubated in an incubator at 37° C. with 5% CO₂ for 22 h. Assays were typically performed in duplicate. Luminescence was measured using a SpectraMax M5 plate reader, after removal of media and addition of the Detection Substrate. Data were normalized to E₂, which had agonist activity IC₅₀ of 0.31±0.03 nM and 0.022±0.005 nM for ERα and ERβ, respectively. Data were fitted to the equation below using GraphPad Prism:

$$y = \frac{bottom + ( top - bottom )}{( 1 + {10}^{( log I {C}_{50} - x ) HillSlope} )}$$

As described for the TR-FRET assay fitting, IC₅₀ values and standard deviations are from the nonlinear least squares fit of the data; and, when replicate assays and fits were done, median values were reported in Table 1.

In Vitro Druggability Assays—CYP450 Binding, hERG & Nephelometry. The P450-Glo™ Screening System from Promega Corporation (Madison, Wis.) was used to measure CYP450 (cytochrome P450) inhibition, as described in the kit instructions. Assays were run in 96-well white plates (Corning® 3912), and luminescence was measured on a SpectraMax M5 instrument (FIG. 6). The luminescence signal is proportional to the amount of luciferin product formed by the CYP reaction. Compounds were prepared in DMSO, then an eight-step 1:2 dilution series was made in DMSO. This was diluted in water such that the DMSO in the assay did not exceed 0.25% and the highest final concentration of compound was 62.6 μM. After adding the relevant Cytochrome P450 enzyme, the plate was incubated at 37° C. for 10 minutes to allow components to come to temperature. Next, the NADPH regeneration system was added to activate the reaction on a luminogenic P450-Glo™ substrate, and incubated at 37° C. for 10-30 min, according to kit instructions for each CYP enzyme. The enzyme reaction was stopped by the addition of Luciferin Detection Reagent and luminescence of the plate was read in a Spectramax M5 (Molecular Devices) after a 20 min incubation at room temperature. Data were normalized to positive controls (α-naphthoflavone for CYP1A2, sulfaphenazole for CYP2C9, quinidine for CYP2D6, and ketoconazole for CYP3A4). Data analysis was with Prism software, as described above.

Nephelometry was performed to determine the relative propensity of compounds to aggregate in solution (FIG. 11a), based on the light scattering properties of the molecular aggregates. Compound aggregation in solution is important to measure in screening campaigns, as aggregation is a common source of artifactual activity; and, it provides a measure of compound solubility. Compounds were tested for aggregation in clear 96-well plates (Greiner BioOne). Progesterone was used as a positive control for compound aggregation. Data were collected using a BMG NEPHELOStar Plus, equipped with a 635 nm laser.

hERG assays were performed using the SelectScreen service from ThermoFisher (FIG. 11b). The assay is a fluorescence polarization assay that measures displacement of a fluorescently tagged Predictor™ tracer, as described.⁴⁷

MTT Assays. Human breast cancer cells (MCF-7) were provided by Dr. Manish Patankar (Department of Obstetrics and Gynecology, University of Wisconsin-Madison). Cells were cultured in Eagle's Minimum Essential Media (EMEM) supplemented with 10% fetal bovine serum and 0.01 mg/mL human recombinant insulin in 5% CO₂ at 37° C. A seeding density of 7,000 cells per well was chosen and applied to a 96 well plate. After 24 hours, treatments of ISP358-2, DPN or estradiol in media containing 0.1% Dimethyl sulfoxide (DMSO), were applied to the cells at varying concentrations (10, 1, 0.1, 0.01, and 0.001 μM). Negative, positive and untreated control cells received 100% DMSO, 0.01 μM estradiol in EMEM or EMEM with 0.1% DMSO content, respectively. Treated cells were incubated for 24 hours after which an MTT (3-(4,5-Dimethylthiazol-2-yl)-2,5-Diphenyltetrazolium Bromide) assay was performed by adding 20% MTT in EMEM solution to each well and incubating for 4 hours. Formazan crystal metabolites were dissolved using 100% DMSO and absorbance was read at OD 570 nm as well as a reference of 650 nm using a VMax kinetic microplate reader (Molecular Devices, CA) running Softmax Pro version 6.1. Absorbances were converted to cell number using a standard growth curve. Two-sample equal variance t-tests were conducted using Microsoft Excel to determine if cell proliferation was significantly different from untreated controls or cells treated with 0.1 μM E₂.

Docking. Three dimensional (3D) conformations were prepared for all ligands, before docking (FIG. 7). AutoDock Tools (ADT) version 1.5.6 was used prepare the ligand files for subsequent AutoDock calculations and assign Gasteiger charges. The ERα receptor for agonist (pdb code 1ere)⁴⁸ and antagonist (pdb code 1err)⁴⁹ conformations were prepared for docking calculations; and, the ERβ receptor for agonist (pdb code 2jj3)⁵⁰ and antagonist (pdb code 112j)⁵¹ conformations were also prepared for docking calculations. ADT was used to add hydrogen atoms and partial charges to each atom of the protein. The grid box was centered on the co-crystallized ligand, drawn to a box to incorporate active site amino acids (Arg394, Glu353, and His524 for ERα and Arg346, Glu305, and His475 for ERβ), and the estradiol ligand was removed.⁵² AutoDock Vina⁵³ was used for docking calculations, with default parameters, except that an energy range of 4 and exhaustiveness of 8 were used.^{47, 54-57} As a control experiment, 17β-estradiol was docked into the structure of ERα (pdb code 1ERE), after removing 17β-estradiol, and found to adopt the same binding mode as for the originally bound 17β-estradiol (data not shown).

Assessment of Memory Consolidation. Subjects. C57BL/6 female mice (8-10 weeks of age) were purchased from Taconic Biosciences. Mice were singly housed in a 12 h light/dark cycle room, with food and water ad libitum. All procedures with live mice were performed between 9:00 am and 6:00 pm in a room with a light intensity of dimmer than 100 lux. All procedures were approved by the University of Wisconsin-Milwaukee Institutional Animal Care and Use Committee and observed policies of the National Institutes of Health Guide for the Care and Use of Laboratory Animals.

General experimental design. A series of three experiments were conducted in mice that were bilaterally ovariectomized to remove the primary source of circulating estrogens. In each experiment, a negative control (dimethylsulfoxide, DMSO), positive control (2,3-bis(4-hydroxyphenyl)-propionitrile, DPN), and multiple doses of ISP358-2 were administered to separate groups of mice via one of three routes of administration: direct bilateral dorsal hippocampal infusion, intraperitoneal injection, or oral gavage. All drugs were administered acutely immediately after training in object recognition and object locations tasks designed to test hippocampal-dependent object recognition and spatial memory consolidation, respectively, as described below (FIG. 8).

Surgery. Four days after arrival in the laboratory, mice were bilaterally ovariectomized as described previously.^{34, 58, 59} Mice slated to receive dorsal hippocampal infusion of ISP358-2 were also implanted with guide cannulae into the dorsal hippocampus (DH) as described previously.³⁰⁻³² Mice were anesthetized with isoflurane gas (2% isoflurane in 100% oxygen) and placed in a stereotaxic apparatus (Kopf Instruments) Immediately after ovariectomy, mice were implanted with two guide cannulae (22 gauge; C232G, Plastics One) aimed at the dorsal hippocampus (−1.7 mm AP, ±1.5 mm ML, −2.3 mm DV). Dummy cannulae (C232DC, Plastics One) were placed inside the guide cannulae to conserve patency of the guide cannulae. Dental cement (Darby Dental) was applied to anchor the guide cannulae to the skull and also served to close the wound. Mice were allowed to recover for six days before behavioral testing.

Drugs and infusions. Dorsal hippocampal (DH) infusions or intraperitoneal (IP) injections were conducted immediately post-training as described previously.^{34, 58, 59} During infusions, mice were gently restrained and drugs delivered using an infusion cannula (C3131, 28-gauge, extending 0.8 mm beyond the 1.5 mm guide). The infusion cannula was connected to a 10 μl Hamilton syringe using PE20 polyethylene tubing. The infusion was controlled by a microinfusion pump (KDS Legato 180, KD Scientific) at a rate of 0.5 μl/minute. Each infusion was followed by a one-minute waiting period to prevent diffusion back up the cannula track and allow the drug to diffuse through the tissue. The negative control (“vehicle”) was 1% DMSO in 0.9% saline. As a positive control, the ERβ agonist DPN (2,3-bis(4-hydroxyphenyl)-propionitrile, Tocris Bioscience) was dissolved in 1% DMSO in saline and infused at a dose of 10 pg/hemisphere.³⁰ DPN has a 70-fold higher affinity for ERβ than ERα,⁶⁰ and bilateral infusion of 10 pg/hemisphere into the dorsal hippocampal previously enhanced memory consolidation in the object recognition and object placement tasks in young adult ovariectomized mice.³⁴ ISP358-2 was dissolved in 1% DMSO to a concentration of 2 ng/μl and then diluted to administer doses of 1 ng/hemisphere, 100 pg/hemisphere, and 10 pg/hemisphere.

For intraperitoneal injections, ISP358-2 was dissolved in 10% DMSO in physiological saline and injected at doses of 0.5 or 5 mg/kg in a volume of 10 ml/kg. DPN was dissolved in 10% DMSO in saline and injected at a dose of 0.05 mg/kg in volume of 10 ml/kg. This dose previously enhanced object recognition memory consolidation in young adult ovariectomized mice.³¹ Vehicle controls received 10 ml/kg of 10% DMSO in saline. For oral gavage, all drugs were administered in a volume of 10 ml/kg at the same doses as intraperitoneal injections; 0.5 or 5 mg/kg ISP358-2 and 0.05 mg/kg DPN. Vehicle controls received 10% DMSO in saline. In the procedure, a bulb tipped gastric gavage needle (24 GA, 25 mm) was used to deliver the drugs directly to the stomach.

Memory testing. Object recognition and object placement were performed as described previously.^{34, 58, 59} Object recognition and object placement evaluated object recognition memory and spatial memory, respectively, and require intact dorsal hippocampal function.^{39, 61-63} Mice were first handled (30 sec/d) for 3 days to acclimate them to the experimenters. On the second day of handling, a small Lego was placed in the home cage to habituate the mice to objects. This Lego was removed from the cage just before training. After 3 days of handling, mice were habituated to an empty white arena (width, 60 cm; length, 60 cm; height, 47 cm) by allowing them to explore freely for 5 min each day for two days. On the training day, mice were habituated for 2 min in the arena, and then removed to their home cage. Two identical objects were then placed near the northwest and northeast corners of the arena. Mice were returned to the arena allowed to explore until they accumulated a total of 30 s exploring the objects (or until a total of 20 min had elapsed) Immediately after this training, mice were removed from the arena, infused, and then returned to their home cage. Object placement memory was tested 24 h after training by moving one of the training objects to the southeast or southwest corner of the box. Because mice inherently prefer novelty, mice that remember the location of the training objects spend more time with moved object than the unmoved object. Mice performing at chance (15 s) spend an equal amount of time with each object and demonstrate no memory consolidation. Thus, consolidation of memory for the training objects is demonstrated if mice spend significantly more time than chance with the moved object. Object recognition training was conducted two weeks after object placement. The object recognition task used the same apparatus and general procedure as object placement, but instead of changing the object location, one familiar object was replaced with a new object during testing. Object recognition testing occurred 48 h after training. As with object placement, mice accumulated 30 s exploring the novel and familiar objects. Because mice are inherently drawn to novelty, more time than chance spent exploring the novel object indicated memory for the familiar training object. To maintain novelty, different objects were used in the object placement and object recognition tasks. Because vehicle-infused female mice do not remember the location of the training objects 24 h after training,³⁴ a 24-h delay was used to test the memory-enhancing effects of drugs in object placement. Similarly, because vehicle-infused female mice do not remember the familiar object 48 h after training,³⁴ a 48-h delay was used to test the memory-enhancing effects of drugs in object recognition. For both tasks, the time spent exploring each object and elapsed time to accumulate 30 s of exploration were recorded using ANYmaze tracking software (Stoelting).

Behavioral data analysis. One-sample t-tests and one-way analyses of variance (ANOVAs) were conducted using GraphPad Prism 6 (La Jolla, Calif.). One-sample t-tests were used to determine whether mice spent significantly more time than chance (15 s) investigating the novel or moved object, indicating whether each group of mice successfully formed a memory of the identity and location of the training objects. To determine the extent to which DPN or ISP358-2 treatment influenced memory consolidation relative to vehicle, between-group comparisons were conducted for each behavioral task using one-way ANOVAs, followed by Fisher's LSD post hoc tests. Significance was determined at p>0.05.

Assessment of Potential Peripheral Pathology. To assess possible toxicity of ISP358-2 treatment to peripheral organs, ovariectomized mice received a single intraperitoneal injection of vehicle or ISP358-2, and liver, kidney, and heart tissues were collected 24 hours later. Similar to behavioral testing, ISP358-2 was injected at doses of 0.5 or 5 mg/kg in a volume of 10 ml/kg and DPN was injected at a dose of 0.05 mg/kg in a volume of 10 ml/kg. Vehicle controls received 10 ml/kg of 10% DMSO in saline (FIG. 13a). Tissues were fixed in 10% formalin buffered solution for 24 hours. Twenty specimens were processed and analyzed. Each specimen contained three pieces of tissue. The tissues from each animal was transferred to a labeled cassette and processed on an automated tissue processor following standard procedures. The tissues were then embedded in paraffin wax. No specific orientation of the tissue was performed. Four-micron sections were cut from each paraffin block and placed onto a slide. The slides were then stained using hematoxylin and eosin (H&E) on an automated stainer (FIG. 13b).

The slides were then examined by a pathologist (ACM) who is board certified in anatomical pathology by the American Board of Pathology. All specimens contained three tissue samples corresponding to liver, kidney, and heart. In some instances, portions of adjacent tissues were also present. For example, several specimens had gall bladder. One specimen had a portion of spleen. Each organ was examined for specific pathological changes. Three major categories of change were examined: (1) structural changes to the organs. For liver, the central vein, portal triads, and hepatocytes were examined. For kidneys, the glomeruli and tubules were examined. For heart, the myocytes and coronary vessels were examined, (2) evidence of inflammation was evaluated including hepatitis, glomerulonephritis, interstitial nephritis, and myocarditis, and (3) evidence of ischemic changes was examined See the attached table for a summary of the findings.

### REFERENCES

1. Manas, E. S.; Unwalla, R. J.; Xu, Z. B.; Malamas, M. S.; Miller, C. P.; Harris, H. A.; Hsiao, C.; Akopian, T.; Hum, W. T.; Malakian, K.; Wolfrom, S.; Bapat, A.; Bhat, R. A.; Stahl, M. L.; Somers, W. S.; Alvarez, J. C. Structure-based design of estrogen receptor-beta selective ligands. J Am Chem Soc 2004, 126, 15106-15119.
2. Malamas, M. S.; Manas, E. S.; McDevitt, R. E.; Gunawan, I.; Xu, Z. B.; Collini, M. D.; Miller, C. P.; Dinh, T.; Henderson, R. A.; Keith, J. C., Jr.; Harris, H. A. Design and synthesis of aryl diphenolic azoles as potent and selective estrogen receptor-beta ligands. J Med Chem 2004, 47, 5021-5040.
3. Liu, F.; Day, M.; Muniz, L. C.; Bitran, D.; Arias, R.; Revilla-Sanchez, R.; Grauer, S.; Zhang, G.; Kelley, C.; Pulito, V.; Sung, A.; Mervis, R. F.; Navarra, R.; Hirst, W. D.; Reinhart, P. H.; Marquis, K. L.; Moss, S. J.; Pangalos, M. N.; Brandon, N. J. Activation of estrogen receptor-beta regulates hippocampal synaptic plasticity and improves memory. Nat Neurosci 2008, 11, 334-343.
4. www.clinicaltrials.gov (last visited Oct. 8, 2017)
5. Sarvari, M.; Kallo, I.; Hrabovszky, E.; Solymosi, N.; Rodolosse, A.; Liposits, Z. Long-term estrogen receptor beta agonist treatment modifies the hippocampal transcriptome in middle-aged ovariectomized rats. Front Cell Neurosci 2016, 10, 149.
6. Frick, K. M.; Kim, J.; Tuscher, J. J.; Fortress, A. M. Sex steroid hormones matter for learning and memory: estrogenic regulation of hippocampal function in male and female rodents. Learn Mem 2015, 22, 472-493.
7. Tian, Z.; Fan, J.; Zhao, Y.; Bi, S.; Si, L.; Liu, Q. Estrogen receptor beta treats Alzheimer's disease. Neural Regen Res 2013, 8, 420-426.
8. Chen, L. H.; Fan, Y. H.; Kao, P. Y.; Ho, D. T.; Ha, J. C.; Chu, L. W.; Song, Y. Q. Genetic polymorphisms in estrogen metabolic pathway associated with risks of alzheimer's disease: Evidence from a Southern Chinese Population. J Am Geriatr Soc 2017, 65, 332-339.
9. Farrer, L. A.; Cupples, L. A.; Haines, J. L.; Hyman, B.; Kukull, W. A.; Mayeux, R.; Myers, R. H.; Pericak-Vance, M. A.; Risch, N.; van Duijn, C. M. Effects of age, sex, and ethnicity on the association between apolipoprotein E genotype and Alzheimer disease. A meta-analysis. APOE and Alzheimer Disease Meta Analysis Consortium. JAMA 1997, 278, 1349-1356.
10. Altmann, A.; Tian, L.; Henderson, V. W.; Greicius, M. D. Alzheimer's disease Neuroimaging Initiative, I. Sex modifies the APOE-related risk of developing Alzheimer disease. Ann Neurol 2014, 75, 563-573.
11. Bretsky, P. M.; Buckwalter, J. G.; Seeman, T. E.; Miller, C. A.; Poirier, J.; Schellenberg, G. D.; Finch, C. E.; Henderson, V. W. Evidence for an interaction between apolipoprotein E genotype, gender, and Alzheimer disease Alzheimer Dis Assoc Disord 1999, 13, 216-221.
12. Holmes, S. E.; Esterlis, I.; Mazure, C. M.; Lim, Y. Y.; Ames, D.; Rainey-Smith, S.; Martins, R. N.; Salvado, O.; Dore, V.; Villemagne, V. L.; Rowe, C. C.; Laws, S. M.; Masters, C. L.; Maruff, P.; Pietrzak, R. H.; Australian Imaging, B. L. R. G. beta-Amyloid, APOE and BDNF genotype, and depressive and anxiety symptoms in cognitively normal older women and men. Am J Geriatr Psychiatry 2016, 24, 1191-1195.
13. Brinton, R. D. Cellular and molecular mechanisms of estrogen regulation of memory function and neuroprotection against Alzheimer's disease: recent insights and remaining challenges. Learn Mem 2001, 8, 121-133.
14. Gartlehner, G.; Patel, S. V.; Feltner, C.; Weber, R. P.; Long, R.; Mullican, K.; Bolandm E.; Lux, L.; Viswanathan, M. Hormone replacement therapy for the primary prevention of chronic conditions in postmenopausal women: evidence report and a systematic review of the US preventive services task force. JAMA 2017, 318, 2234-2249.
15. Wang. K.; Li, F.; Chen L.; Lai, Y. M.; Zhang, X.; Li, H. Y. Change in risk of breast cancer after receiving hormone replacement therapy by considering effect-modifiers: a systematic review and dose response meta-analysis of prospective studies. Oncotarget 2017, 8, 81109-81124.
16. Beral, V. Breast cancer and hormone-replacement therapy in the Million Women Study. Lancet 2003, 362, 419-427.
17. Li, C. I.; Malone, K. E.; Porter, P. L.; Weiss, N. S.; Tang, M. T.; Cushing-Haugen, K. L.; Daling, J. R. Relationship between long durations and different regimens of hormone therapy and risk of breast cancer. JAMA 2003, 289, 3254-3263.
18. Anderson, G. L.; Limacher, M.; Assaf, A. R.; Bassford, T.; Beresford, S. A.; Black, H.; et al. Effects of conjugated equine estrogen in postmenopausal women with hysterectomy: the Women's Health Initiative randomized controlled trial. JAMA 2004, 291, 1701-17012.
19. McCullough, C.; Neumann, T. S.; Gone, J. R.; He, Z.; Herrild, C.; Wondergem Nee Lukesh, J.; Pandey, R. K.; Donaldson, W. A.; Sem, D. S. Probing the human estrogen receptor-alpha binding requirements for phenolic mono- and di-hydroxyl compounds: a combined synthesis, binding and docking study. Bioorg Med Chem 2014, 22, 303-310.
20. Dabrota, C.; Asim, M.; Choueiri, C.; Gargaun, A.; Korobkov, I.; Butt, A.; Carlson, K. E.; Katzenellenbogen, J. A.; Wright, J. S.; Durst, T. Synthesis and receptor binding in trans-CD ring-fused A-CD estrogens: comparison with the cis-fused isomers. Bioorg Med Chem Lett 2014, 24, 3841-3844.
21. Wright, J. S.; Shadnia, H.; Anderson, J. M.; Durst, T.; Asim, M.; El-Salfiti, M.; Choueiri, C.; Pratt, M. A.; Ruddy, S. C.; Lau, R.; Carlson, K. E.; Katzenellenbogen, J. A.; O'Brien, P. J.; Wan, L. A-CD estrogens. I. Substituent effects, hormone potency, and receptor subtype selectivity in a new family of flexible estrogenic compounds. J Med Chem 2011, 54, 433-448.
22. Asim, M.; El-Salfiti, M.; Qian, Y.; Choueiri, C.; Salari, S.; Cheng, J.; Shadnia, H.; Bal, M.; Christine Pratt, M. A.; Carlson, K. E.; Katzenellenbogen, J. A.; Wright, J. S.; Durst, T. Deconstructing estradiol: removal of B-ring generates compounds which are potent and subtype-selective estrogen receptor agonists. Bioorg Med Chem Lett 2009, 19, 1250-1253.
23. Asim, M.; Klonowska, D.; Choueiri, C.; Korobkov, I.; Carlson, K. E.; Katzenellenbogen, J. A.; Durst, T. BC-spiro-estradiols. Synthesis and estrogen receptor binding affinity of four new estradiol isomers. Bioorg Med Chem Lett 2012, 22, 3713-3717.
24. Buchanan, G. W.; Stothers, J. B.; Wu, S.-T. 13C Nuclear magnetic resonance studies. XII. Conformational effects on 13C shieldings in alicyclic oxygenated compounds. Can J Chem 1969, 47, 3113-3118.
25. Patrick, D. W.; Truesdale, L. K.; Biller, S. A.; Sharpless, K. B. Stereospecific vicinal oxyamination of olefins by alkylimidoosmium compounds. J Org Chem 1978, 43, 2628-2638.
26. Brown, H. C.; Liotta, R.; Brener, L. Hydroboration. 46. The regio- and stereochemistry of the hydroboration of representative cyclic olefins with 9-borabicyclo[3.3.1]nonane. J Am Chem Soc 1977, 99, 3427-3432.
27. Hou, X.-L.; Xie, Q.-C.; Dai, L.-X. Stereoselective metal catalysed hydroboration of 4-substituted 1-methylidenecyclohexanes. J Chem Res (S) 1997, 436-436.
28. CCDC 1571231 contains the supplementary crystallographic data for this paper. These data are provided free of charge by The Cambridge Crystallographic Data Centre.
29. Driscoll, J. P.; Kornecki, K.; Wolkowski, J. P.; Chupak, L.; Kalgutkar, A. S.; O'Donnell, J. P. Bioactivation of phencyclidine in rat and human liver microsomes and recombinant P450 2B enzymes: evidence for the formation of a novel quinone methide intermediate. Chem Res Toxicol 2007, 20, 1488-1497.
30. Inoue, Y.; Kiryu, S.; Izawa, K.; Watanabe, M.; Tojo, A.; Ohtomo, K. Comparison of subcutaneous and intraperitoneal injection of D-luciferin for in vivo bioluminescence imaging. Eur J Nucl Med Mol Imaging 2009, 36, 771-779.
31. Frick, K. M.; Fernandez, S. M.; Harburger, L. L. A new approach to understanding the molecular mechanisms through which estrogens affect cognition. Biochim Biophys Acta 2010, 1800, 1045-1055.
32. Atcha, Z.; Rourke, C.; Neo, A. H.; Goh, C. W.; Lim, J. S.; Aw, C. C.; Browne, E. R.; Pemberton, D. J. Alternative method of oral dosing for rats. J Am Assoc Lab Anim Sci 2010, 49, 335-343.
33. Machholz, E.; Mulder, G.; Ruiz, C.; Corning, B. F.; Pritchett-Corning, K. R. Manual restraint and common compound administration routes in mice and rats. J Vis Exp 2012, pii 2071 (http://www.jove.com/video/2771 Last visited Oct. 16, 2017).
34. Boulware, M. I.; Heisler, J. D.; Frick, K. M. The memory-enhancing effects of hippocampal estrogen receptor activation involve metabotropic glutamate receptor signaling. J Neurosci 2013, 33, 15184-15194.
35. Koehler, K. F.; Helguero, L. A.; Haldosen, L. A.; Warner, M.; Gustafsson, J. A. Reflections on the discovery and significance of estrogen receptor beta. Endocrine reviews. 2005, 26, 465-478.
36. Brzozowski, A. M.; Pike, A. C.; Dauter, Z.; Hubbard, R. E.; Bonn, T.; Engstrom, O.; Ohman, L.; Greene, G. L.; Gustafsson, J. A.; Carlquist, M. Nature. Molecular basis of agonism and antagonism in the oestrogen receptor 1997, 389, 753-758.
37. Stierand, K.; Rarey, M. Drawing the PDB: Protein-ligand complexes in two dimensions. ACS Med. Chem. Lett. 2010, 1, 540-545.
38. Tuscher, J. J.; Fortress, A. M.; Kim, J.; Frick, K. M. Regulation of object recognition and object placement by ovarian sex steroid hormones. Behav Brain Res 2015, 285, 140-157.
39. Frye, C. A.; Duffy, C. K.; Walf, A. A. Estrogens and progestins enhance spatial learning of intact and ovariectomized rats in the object placement task. Neurobiol Learn Mem 2007, 88, 208-216.
40. Jacome, L. F.; Gautreaux, C.; Inagaki, T.; Mohan, G.; Alves, S.; Lubbers, L. S.; Luine, V. Estradiol and ERbeta agonists enhance recognition memory, and DPN, an ERbeta agonist, alters brain monoamines Neurobiol Learn Mem 2010, 94, 488-498.
41. Phan, A.; Lancaster, K. E.; Armstrong, J. N.; MacLusky, N. J.; Choleris, E. Rapid effects of estrogen receptor alpha and beta selective agonists on learning and dendritic spines in female mice. Endocrinology 2011, 152, 1492-1502.
42. Walf, A. A.; Koonce, C. J.; Frye, C. A. Estradiol or diarylpropionitrile administration to wild type, but not estrogen receptor beta knockout, mice enhances performance in the object recognition and object placement tasks. Neurobiol Learn Mem 2008, 89, 513-521.
43. Gillies, G. E.; McArthur, S. Estrogen actions in the brain and the basis for differential action in men and women: a case for sex-specific medicines. Pharmacol Rev 2010, 62, 155-198.
44. Bodo, C.; Rissman, E. F. New roles for estrogen receptor beta in behavior and neuroendocrinology. Front Neuroendocrinol 2006, 27, 217-232.
45. Lund, T. D.; Rovis, T.; Chung, W. C.; Handa, R. J. Novel actions of estrogen receptor-beta on anxiety-related behaviors. Endocrinology 2005, 146, 797-807.
46. Small, S. A.; Schobel, S. A.; Buxton, R. B.; Witter, M. P.; Barnes, C. A. A pathophysiological framework of hippocampal dysfunction in ageing and disease. Nat Rev Neurosci 2011, 12, 585-601
47. Piper, D. R.; Duff, S. R.; Eliason, H. C.; Frazee, W. J.; Frey, E. A.; Fuerstenau-Sharp, M.; Jachec, C.; Marks, B. D.; Pollok, B. A.; Shekhani, M. S.; Thompson, D. V.; Whitney, P.; Vogel, K. W.; Hess, S. D. Development of the predictor HERG fluorescence polarization assay using a membrane protein enrichment approach. Assay Drug Dev Technol 2008, 6, 213-223.
48. Brzozowski, A. M.; Pike, A. C.; Dauter, Z.; Hubbard, R. E.; Bonn, T.; Engstrom, O.; Ohman, L.; Greene, G. L.; Gustafsson, J. A.; Carlquist, M. Molecular basis of agonism and antagonism in the oestrogen receptor. Nature 1997, 389, 753-758.
49. Norman, B. H.; Richardson, T. I.; Dodge, J. A.; Pfeifer, L. A.; Durst, G. L.; Wang, Y.; Durbin, J. D.; Krishnan, V.; Dinn, S. R.; Liu, S.; Reilly, J. E.; Ryter, K. T. Benzopyrans as selective estrogen receptor beta agonists (SERBAs). Part 4: functionalization of the benzopyran A-ring. Bioorg Med Chem Lett 2007, 17, 5082-5085.
50. Shiau, A. K.; Barstad, D.; Radek, J. T.; Meyers, M. J.; Nettles, K. W.; Katzenellenbogen, B. S.; Katzenellenbogen, J. A.; Agard, D. A.; Greene, G. L. Structural characterization of a subtype-selective ligand reveals a novel mode of estrogen receptor antagonism. Nat Struct Biol 2002, 9, 359-364.
51. Tuccinardi, T.; Bertini, S.; Martinelli, A.; Minutolo, F.; Ortore, G.; Placanica, G.; Prota, G.; Rapposelli, S.; Carlson, K. E.; Katzenellenbogen, J. A.; Macchia, M. Synthesis of anthranylaldoxime derivatives as estrogen receptor ligands and computational prediction of binding modes. J Med Chem 2006, 49, 5001-5012.
52. Morris, G. M.; Huey, R.; Lindstrom, W.; Sanner, M. F.; Belew, R. K.; Goodsell, D. S.; Olson, A. J. AutoDock4 and AutoDockTools4: Automated docking with selective receptor flexibility. J Comput Chem 2009, 30, 2785-2791.
53. Cavasotto, C. N.; Orry, A. J. Ligand docking and structure-based virtual screening in drug discovery. Curr Top Med Chem 2007, 7, 1006-1014.
54. Goodsell, D. S.; Morris, G. M.; Olson, A. J. Automated docking of flexible ligands: applications of AutoDock. J Mol Recognit 1996, 9, 1-5.
55. Huey, R.; Morris, G. M.; Olson, A. J.; Goodsell, D. S. A semiempirical free energy force field with charge-based desolvation. J Comput Chem 2007, 28, 1145-1152.
56. Morris, G. M.; Goodsell, D. S.; Halliday, R. S.; Huey, R.; Hart, W. E.; Belew, R. K.; Olson, A. J. Automated docking using a Lamarckian genetic algorithm and an empirical binding free energy function. J Comput Chem 1998, 19, 1639-1662.
57. Li, Z.; Zhang, H.; Gibson, M.; Li, J. An evaluation on combination effects of phenolic endocrine disruptors by estrogen receptor binding assay. Toxicol In Vitro 2012, 26, 769-774.
58. Fortress, A. M.; Fan, L.; Orr, P. T.; Zhao, Z.; Frick, K. M. Estradiol-induced object recognition memory consolidation is dependent on activation of mTOR signaling in the dorsal hippocampus. Learn Mem 2013, 20, 147-155.
59. Kim, J.; Szinte, J. S.; Boulware, M. I.; Frick, K. M. 17beta-estradiol and agonism of G-protein-coupled estrogen receptor enhance hippocampal memory via different cell-signaling mechanisms. J Neurosci 2016, 36, 3309-3321.
60. Meyers, M. J.; Sun, J.; Carlson, K. E.; Marriner, G. A.; Katzenellenbogen, B. S.; Katzenellenbogen, J. A. Estrogen receptor-beta potency-selective ligands: structure-activity relationship studies of diarylpropionitriles and their acetylene and polar analogues. J Med Chem 2001, 44, 4230-4251.
61. Baker, K. B.; Kim, J J Effects of stress and hippocampal NMDA receptor antagonism on recognition memory in rats. Learn Mem 2002, 9, 58-65.
62. Luine, V. N.; Jacome, L. F.; Maclusky, N. J. Rapid enhancement of visual and place memory by estrogens in rats. Endocrinology 2003, 144, 2836-2844.
63 Cohen, S. J.; Munchow, A. H.; Rios, L. M.; Zhang, G.; Asgeirsdottir, H. N.; Stackman, R. W., Jr. The rodent hippocampus is essential for nonspatial object memory. Curr Biol 2013, 23, 1685-1690.
64. Dehring, K. A.; Workman, H. L.; Miller, K. D.; Mandagere, A.; Poole, S. K., Automated robotic liquid handling/laser-based nephelometry system for high throughput measurement of kinetic aqueous solubility. J Pharm Biomed Anal 2004, 36 (3), 447-56.
65. Jamalzadeh, L.; Ghafoori, H.; Sariri, R.; Rabuti, H.; Nasirzade, J.; Hasani, H.; Aghamaali, M. R., Cytotoxic effects of some common organic solvents on MCF-7, RAW-264.7 and human umbilical vein endothelial cells. Avicenna J Med Biochem 2016, 4 (1), e33453.
66. Deroo et al., “Estrogen Receptors and Human Disease,” J. Clin. Invest. 116:561-570(2006).
67. Mueller et al. (2001), “Estrogen receptors and endocrine diseases: lessons from estrogen receptor knockout mice,” Curr. Opin. Pharmacol. 1: 613-619.
68. Hernyk et al., “Estrogen receptor mutations in human disease,” (2004) Endocr. Rev. 25:869-898.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A compound having a formula and stereochemistry as follows:

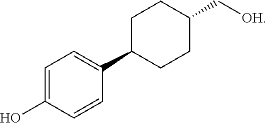 [SMILES: OC[C@H]1CC[C@H](c2ccc(O)cc2)CC1]

2. A pharmaceutical composition comprising an effective amount of the compound of claim 1, or a pharmaceutically acceptable salt thereof, together with a pharmaceutical excipient, carrier, or diluent.

3. A method for treating a disease or disorder associated with estrogen receptor β (ERβ) activity in a subject in need thereof, the method comprising administering to the subject the pharmaceutical composition of claim 2.

4. The method of claim 3, wherein the disease or disorder is a neurological disease or disorder.

5. The method of claim 3, wherein the disease or disorder is a psychiatric disease or disorder.

6. The method of claim 3, wherein the disease or disorder is cancer.

7. The method of claim 3, wherein the disease or disorder is associated with memory loss or memory dysfunction.

8. A method for enhancing memory consolidation in a subject in need thereof, the method comprising administering to the subject the pharmaceutical composition of claim 2.

9. The method of claim 8, wherein the subject is a post-menopausal woman.

10. A method for treating a subject exhibiting low estrogen levels, the method comprising administering to the subject the pharmaceutical composition of claim 2.

11. The method of 10, wherein the subject is a post-menopausal woman.

12. A compound having a formula selected from

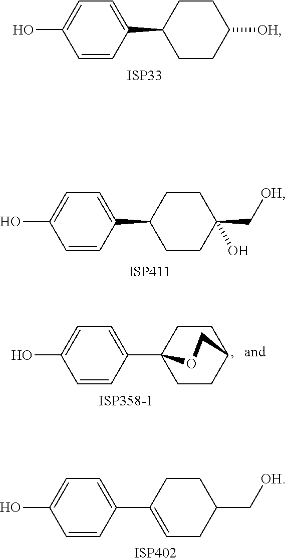

13. A pharmaceutical composition comprising an effective amount of the compound of claim 12, or a pharmaceutically acceptable salt thereof, together with a pharmaceutical excipient, carrier, or diluent.

14.-20. (canceled)

21. A method for enhancing memory consolidation in a subject in need thereof, the method comprising administering to the subject a compound or a pharmaceutical composition comprising the compound having a formula: 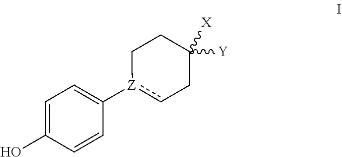
where:
(a) Z is a carbon atom;
(b) X is selected from the group consisting of hydrogen, hydroxyl, alkyl, hydroxyalkyl, amino, and aminoalkyl; and
(c) Y is selected from the group consisting of hydrogen, hydroxyl, alkyl, and hydroxyalkyl; or Y is —CH₂CH₂— or —OCH₂— and Y and Z form a bridge; or X and Y together form alkylidenyl, carboxyalkylidenyl, esteralkylidenyl, hydroxyalkylidenyl, hydroxyalkylalkylidenyl, aminoalkylidenyl, oxo, or oxime.

22. The method of claim 21, wherein the compound has a Formula Ia:

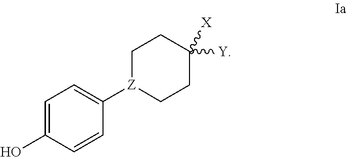

23. The method of claim 21, wherein in the compound X is selected from hydrogen, hydroxyl, alklyl, and hydroxyalkyl; and Y is selected from hydrogen, hydroxyl, alkyl, and hydroxyalkyl; or Y is —OCH₂— and Y and Z form a bridge.

24. The method of claim 21, wherein the compound has a Formula Ia(i):

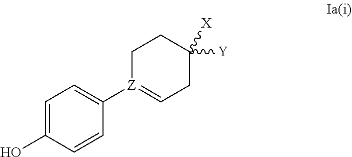

25. The method of claim 24, wherein in the compound X is selected from hydrogen, hydroxyl, alkyl, hydroxylalkyl and Y is hydrogen.

26. The method of claim 21, wherein in the compound X is hydrogen or methyl, and Y is hydroxymethyl (—CH₂OH) or hydroxyethyl (—CH₂CH₂OH).

27. The method of claim 21, wherein in the compound X is methyl, and Y is Y is hydroxymethyl (—CH₂OH).


SEQUENCE LISTING (1 sequence)

SEQ ID NO: 1 - protein, 19 aa, Homo sapiens
EAEEPSLLKKLLLAPANTQ